(19) United States
(12) Patent Application Publication
(10) Pub. No.: US 2013/0090496 A1
(43) Pub. Date: Apr. 11, 2013

(54) PROCESS FOR PREPARING FORMIC ACID BY REACTING CARBON DIOXIDE WITH HYDROGEN

(71) Applicants: Thomas Schaub, Neustadt (DE); Oliver Bey, Niederkirchen (DE); Anton Meier, Birkenheide (DE); Donata Maria Fries, Mannheim (DE); Randolf Hugo, Dirmstein (DE)

(72) Inventors: Thomas Schaub, Neustadt (DE); Oliver Bey, Niederkirchen (DE); Anton Meier, Birkenheide (DE); Donata Maria Fries, Mannheim (DE); Randolf Hugo, Dirmstein (DE)

(73) Assignee: BASF SE, Ludwigshafen (DE)

(21) Appl. No.: 13/646,161

(22) Filed: Oct. 5, 2012

Related U.S. Application Data
(60) Provisional application No. 61/544,291, filed on Oct. 7, 2011.

(51) Int. Cl. C07C 51/15

(52) U.S. Cl. 562/609

(57) ABSTRACT

The invention relates to a process for preparing formic acid by reacting carbon dioxide with hydrogen in a hydrogenation reactor in the presence of a catalyst comprising an element of group 8, 9 or 10 of the Periodic Table, a tertiary amine and a polar solvent to form formic acid-amine adducts which are subsequently dissociated thermally into formic acid and tertiary amine.

This patent application claims the benefit of pending U.S. provisional patent application Ser. No. 61/544,291, filed Oct. 7, 2011, incorporated in its entirety herein by reference.

The invention relates to a process for preparing formic acid by reacting carbon dioxide with hydrogen in a hydrogenation reactor in the presence of a catalyst comprising an element of group 8, 9 or 10 of the Periodic Table, a tertiary amine and a polar solvent to form formic acid-amine adducts which are subsequently dissociated thermally into formic acid and tertiary amine.

Adducts of formic acid and tertiary amines can be dissociated thermally into free formic acid and tertiary amine and therefore serve as intermediates in the preparation of formic acid.

Formic acid is an important and versatile product. It is used, for example, for acidification in the production of animal feeds, as preservative, as disinfectant, as auxiliary in the textile and leather industry, as a mixture with its salts for deicing aircraft and runways and also as synthetic building block in the chemical industry.

The abovementioned adducts of formic acid and tertiary amines can be prepared in various ways, for example (i) by direct reaction of the tertiary amine with formic acid, (ii) by hydrolysis of methyl formate to formic acid in the presence of the tertiary amine, (iii) by catalytic hydration of carbon monoxide in the presence of the tertiary amine or (iv) by hydrogenation of carbon dioxide to formic acid in the presence of the tertiary amine. The last-named process of catalytic hydrogenation of carbon dioxide has the particular advantage that carbon dioxide is available in large quantities and is flexible in terms of its source.

EP 0 181 078 describes a process for preparing formic acid by thermal dissociation of adducts of formic acid and a tertiary amine. According to EP 0 181 078, the process for preparing formic acid comprises the following steps:
i) reaction of carbon dioxide and hydrogen in the presence of a volatile tertiary amine and a catalyst to give the adduct of formic acid and the volatile tertiary amine,
ii) separation of the adduct of formic acid and volatile tertiary amine from the catalyst and the gaseous components in an evaporator,
iii) separation of the unreacted volatile tertiary amine from the adduct of formic acid and the volatile tertiary amine in a distillation column or in a phase separation apparatus,
iv) base exchange of the volatile tertiary amine in the adduct of formic acid and the volatile tertiary amine by a less volatile and weaker nitrogen base, for example 1-n-butylimidazole,
v) thermal dissociation of the adduct of formic acid and the less volatile and weaker nitrogen base to give formic acid and the less volatile and weaker nitrogen base.

In EP 0 181 078, the volatile tertiary amine in the formic acid adduct must be replaced by a less volatile and weaker nitrogen base, for example 1-n-butylimidazole, before the thermal dissociation. The process according to EP 0 181 078 is therefore very complicated, especially in respect of the base exchange which is absolutely necessary.

A further significant disadvantage of the process according to EP 0 181 078 is the fact that the isolation of the adduct of formic acid and volatile tertiary amine is carried out in an evaporator in the presence of the catalyst in accordance with the above-described step ii) of EP 0 181 078.

This catalyzes the redissociation of the adduct of formic acid and volatile tertiary amine into carbon dioxide, hydrogen and volatile tertiary amine according to the following reaction equation:

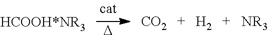

The redissociation leads to a significant decrease in the yield of adduct of formic acid and volatile tertiary amine and thus to a reduction in the yield of the target product formic acid.

In EP 0 329 337 the addition of an inhibitor which inhibits the catalyst is proposed as a solution to this problem. As preferred inhibitors, mention is made of carboxylic acids, carbon monoxide and oxidants. The preparation of formic acid therefore comprises the steps i) to v) described above in EP 0 181 078, with the addition of the inhibitor being carried out after step i) and before or during step ii).

Disadvantages of the process according to EP 0 329 337 are not only the complicated base exchange (step iv)) but also the fact that the inhibitor goes together with the recirculated tertiary amine into the hydrogenation (step (i)), if carboxylic acids are used as inhibitors, and there interferes in the synthesis to form the adduct of formic acid and volatile tertiary amine. When carbon monoxide and oxidants are used, reversible inhibition of the catalyst is indeed possible according to the process according to EP 0 329 337, and the catalyst can be recirculated to the reaction. A basic disadvantage of EP 0 329 337 is, however, that a major part of the catalyst present in the process is inhibited. A major part of the inhibited catalyst therefore has to be reactivated in an external step in the process according to EP 0 329 337 before renewed use in the hydrogenation (step i)). This requires a large amount of inhibiting agent and high energy input and time expenditure to reactivate the inhibited catalyst.

In addition, the entire bottoms from the separation of the low boilers have to be recirculated to the hydrogenation in order to avoid catalyst losses in the process according to EP 0 329 337. In order to prevent a deterioration in the space-time yield of the hydrogenation, complete evaporation of the formic acid from the bottoms is, moreover, essential.

WO 2010/149507 describes a process for preparing formic acid by hydrogenation of carbon dioxide in the presence of a tertiary amine, a transition metal catalyst and a high-boiling polar solvent having an electrostatic factor of ≧200*10⁻³⁰ Cm, for example ethylene glycol, diethylene glycol, triethylene glycol, polyethylene glycol, 1,3-propanediol, 2-methyl-1,3-propanediol, 1,4-butanediol, dipropylene glycol, 1,5-pentanediol, 1,6-hexanediol and glycerol. A reaction mixture comprising the formic acid-amine adduct, the tertiary amine, the high-boiling polar solvent and the catalyst is obtained. The reaction mixture is, according to WO 2010/149507, worked up according to the following steps:
1) phase separation of the reaction mixture to give an upper phase comprising the tertiary amine and the catalyst and a lower phase comprising the formic acid-amine adduct, the high-boiling polar solvent and catalyst residues; recirculation of the upper phase to the hydrogenation,
2) extraction of the lower phase with the tertiary amine to give an extract comprising the tertiary amine and catalyst residues and a raffinate comprising the high-boiling polar solvent and the formic acid-amine adduct; recirculation of the extract to the hydrogenation,
3) thermal dissociation of the raffinate in a distillation column to give a distillate comprising the formic acid and a bottoms mixture comprising the free tertiary amine and the high-boiling polar solvent; recirculation of the high-boiling polar solvent to the hydrogenation.

The process of WO 2010/149507 has the advantage over the processes of EP 0 181 078 and EP 0 329 337 that it makes do without the complicated base exchange step (step (iv)) and allows isolation and recirculation of the catalyst in its active form.

However, the process of WO 2010/149507 has the disadvantage that the isolation of the catalyst is not always complete despite the phase separation (step 1)) and extraction (step 2)), so that traces of catalyst comprised in the raffinate can, in the thermal dissociation in the distillation column in step 3), catalyze the redissociation of the formic acid-amine adduct into carbon dioxide and hydrogen and the tertiary amine. A further disadvantage is that in the thermal dissociation of the formic acid-amine adduct in the distillation column, esterification of the formic acid formed with the high-boiling polar solvents (diols and polyols) occurs. This leads to a reduction in the yield of the target product formic acid.

It was an object of the present invention to provide a process for preparing formic acid by hydrogenating carbon dioxide, which process does not have the above-mentioned disadvantages of the prior art or has them only to a significantly reduced extent and leads to concentrated formic acid in high yield and high purity. Furthermore, the process should be carried out more simply than described in the prior art, in particular should allow a simpler process concept for the work-up of the output from the hydrogenation reactor, simpler process steps, a lower number of process steps or simpler apparatuses. Furthermore, the process should also be able to be carried out with a very low energy consumption and use of additives such as inhibitors. Since complete separation of the homogeneously dissolved active catalyst from the product stream can be achieved only with a very high outlay and even small amounts of catalyst in the thermal dissociation would lead to significant losses of formic acid because of the high temperatures, it should also be ensured that traces of catalyst are converted into inactive species before the distillation, without the hydrogenation being adversely affected.

The object is achieved by a process for preparing formic acid, which comprises the steps
(a) homogeneously catalyzed reaction of a reaction mixture (Rg) comprising carbon dioxide, hydrogen, at least one polar solvent selected from the group consisting of methanol, ethanol, 1-propanol, 2-propanol, 1-butanol, 2-butanol, 2-methyl-1-propanol and water and also at least one tertiary amine of the general formula (A1)

NR¹R²R³ (A1),
where
R¹, R², R³ are each, independently of one another, an unbranched or branched, acyclic or cyclic, aliphatic, araliphatic or aromatic radical having in each case from 1 to 16 carbon atoms, where individual carbon atoms may, independently of one another, also be replaced by a heterogroup selected from among the groups —O— and >N— and two or all three radicals can also be joined to one another to form a chain comprising at least four atoms,
in the presence of at least one complex catalyst comprising at least one element selected from groups 8, 9 and 10 of the Periodic Table,
in a hydrogenation reactor
to give, optionally after addition of water, a two-phase hydrogenation mixture (H) comprising
an upper phase (U1), which comprises the at least one complex catalyst and the at least one tertiary amine (A1) and
a lower phase (L1) which comprises the at least one polar solvent, residues of the at least one complex catalyst and also at least one formic acid-amine adduct of the general formula (A2),

NR¹R²R³*xᵢHCOOH (A2),
where
xᵢ is in the range from 0.4 to 5 and
R¹, R², R³ are as defined above,
(b) work-up of the hydrogenation mixture (H) obtained in step (a) according to one of the following steps
   (b1) phase separation of the hydrogenation mixture (H) obtained in step (a) into the upper phase (U1) and the lower phase (L1) in a first phase separation apparatus
      or
   (b2) extraction of the at least one complex catalyst from the hydrogenation mixture (H) obtained in step (a) by means of an extractant comprising at least one tertiary amine (A1) in an extraction unit to give
      a raffinate (R1) comprising the at least one formic acid-amine adduct (A2) and the at least one polar solvent and
      an extract (E1) comprising the at least one tertiary amine (A1) and the at least one complex catalyst
      or
   (b3) phase separation of the hydrogenation mixture (H) obtained in step (a) into the upper phase (U1) and the lower phase (L1) in a first phase separation apparatus and extraction of the residues of the at least one complex catalyst from the lower phase (L1) by means of an extractant comprising the at least one tertiary amine (A1) in an extraction unit to give
      a raffinate (R2) comprising the at least one formic acid-amine adduct (A2) and the at least one polar solvent and
      an extract (E2) comprising the at least one tertiary amine (A1) and the residues of the at least one complex catalyst,
(c) separation of the at least one polar solvent from the lower phase (L1), from the raffinate (R1) or from the raffinate (R2) in a first distillation unit to give
   a distillate (D1) comprising the at least one polar solvent, which is recirculated to the hydrogenation reactor in step (a), and
   a two-phase bottoms mixture (S1) comprising
   an upper phase (U2) which comprises the at least one tertiary amine (A1) and a lower phase (L2) which comprises the at least one formic acid-amine adduct (A2),
(d) optionally work-up of the bottoms mixture (S1) obtained in step (c) by phase separation in a second phase separation apparatus to give the upper phase (U2) and the lower phase (L2),
(e) dissociation of the at least one formic acid-amine adduct (A2) comprised in the bottoms mixture (S1) or optionally in the lower phase (L2) in a thermal dissociation unit to give the at least one tertiary amine (A1), which is recirculated to the hydrogenation reactor in step (a), and formic acid, which is discharged from the thermal dissociation unit,
   wherein carbon monoxide is added to the lower phase (L1), the raffinate (R1) or the raffinate (R2) directly before and/or during step (c)
   and/or
   carbon monoxide is added to the bottoms mixture (S1) or optionally to the lower phase (L2) directly before and/or during step (e).

It has been found that formic acid can be obtained in high yield by means of the process of the invention. It is particularly advantageous that the base exchange (step (iv)) as per the processes of EP 0 329 337 and EP 0 181 078 can be saved in the process of the invention. The process of the invention allows effective isolation of the complex catalyst in its active form and also recirculation of the complex catalyst which has been separated off to the hydrogenation reactor in step (a). In addition, the use of an inhibitor prevents the redissociation of the formic acid-amine adduct (A2), which leads to an increase in the formic acid yield. In addition, the process of the invention makes it possible to recirculate a major part of the complex catalyst to the hydrogenation in its active form, so that only small amounts of inhibitor have to be added and thus only a small part of the complex catalyst has to be reactivated again after having been inhibited. Furthermore, the complex catalyst which has been inhibited by means of carbon monoxide in the thermal dissociation can be recirculated via the amine phase from the thermal dissociation unit in step (e) to the hydrogenation in step (a) and is there reactivated again under reaction conditions. In addition, it is not necessary to recirculate the entire bottoms from the thermal dissociation to step (a) in order to avoid catalyst losses in the process of the invention. This has the advantage that the formic acid does not have to be evaporated completely from the bottoms from the thermal dissociation in order to prevent a deterioration in the space-time yield in the hydrogenation, since the bottoms from the thermal dissociation is a two-phase mixture. Phase separation makes it possible to separate off the amine phase which comprises the inhibited complex catalyst and recirculation thereof to the hydrogenation. The formic acid-comprising phase can be returned to the thermal dissociation.

It is also possible to reactivate the inhibited complex catalyst by means of a preceding thermal treatment of the amine phase. Furthermore, the removal of the polar solvent used according to the invention prevents esterification of the formic acid obtained in the thermal dissociation unit in step (e), which likewise leads to an increase in the formic acid yield. In addition, it has surprisingly been found that the use of the polar solvent according to the invention leads to an increase in the concentration of the formic acid-amine adduct (A2) in the hydrogenation mixture (H) obtained in step (a) compared to the high-boiling polar solvents used in WO2010/149507. This makes the use of smaller reactors possible, which in turn leads to a cost saving.

The terms “step” and “process step” are used synonymously in the following.

### Preparation of the Formic Acid-Amine Adduct (A2); Process Step (a)

In process step (a) of the process of the invention, a reaction mixture (Rg) which comprises carbon dioxide, hydrogen, at least one complex catalyst comprising at least one element selected from groups 8, 9 and 10 of the Periodic Table, at least one polar solvent selected from the group consisting of methanol, ethanol, 1-propanol, 1-butanol, 2-butanol, 2-methyl-1-propanol and water and also at least one tertiary amine of the general formula (A1) is reacted in a hydrogenation reactor.

The carbon dioxide used in process step (a) can be solid, liquid or gaseous. It is also possible to use industrially available gas mixtures comprising carbon dioxide, as long as these are largely free of carbon monoxide (proportion by volume of <1% of CO). The hydrogen used in the hydrogenation of carbon dioxide in process step (a) is generally gaseous. Carbon dioxide and hydrogen can also comprise inert gases such as nitrogen or noble gases. However, the content of these is advantageously below 10 mol %, based on the total amount of carbon dioxide and hydrogen in the hydrogenation reactor. Although large amounts may likewise be tolerable, they generally result in the use of a higher pressure in the reactor, which makes further compression energy necessary.

Carbon dioxide and hydrogen can be introduced as separate streams into process step (a). It is also possible to use a mixture comprising carbon dioxide and hydrogen in process step (a).

In the process of the invention, at least one tertiary amine (A1) is used in the hydrogenation of carbon dioxide in process step (a). For the purposes of the present invention, the term “tertiary amine (A1)” refers to both one tertiary amine (A1) and also mixtures of two or more tertiary amines (A1).

The tertiary amine (A1) used in the process of the invention is preferably selected or matched to the polar solvent in such a way that the hydrogenation mixture (H) obtained in process step (a), optionally after addition of water, is an at least two-phase mixture. The hydrogenation mixture (H) comprises an upper phase (U1), which comprises the at least one complex catalyst and the at least one tertiary amine (A1), and a lower phase (L1), which comprises the at least one polar solvent, residues of the complex catalyst and at least one formic acid-amine adduct (A2).

The tertiary amine (A1) should be enriched in the upper phase (U1), i.e. the upper phase (U1) should comprise the major part of the tertiary amine (A1). For the purposes of the present invention, “enriched” or “major part” in respect of the tertiary amine (A1) means a proportion by weight of the free tertiary amine (A1) in the upper phase (U1) of >50% based on the total weight of the free tertiary amine (A1) in the liquid phases, i.e. the upper phase (U1) and the lower phase (L1) in the hydrogenation mixture (H).

For the present purposes, free tertiary amine (A1) is the tertiary amine (A1) which is not bound in the form of the formic acid-amine adduct (A2).

The proportion by weight of the free tertiary amine (A1) in the upper phase (U1) is preferably >70%, in particular >90%, in each case based on the total weight of the free tertiary amine (A1) in the upper phase (U1) and the lower phase (L1) in the hydrogenation mixture (H).

The tertiary amine (A1) is generally selected by a simple test in which the phase behavior and the solubility of the tertiary amine (A1) in the liquid phases (upper phase (U1) and lower phase (L1)) are determined experimentally under the process conditions in process step (a). In addition, nonpolar solvents such as aliphatic, aromatic or araliphatic solvents can be added to the tertiary amine (A1). Preferred nonpolar solvents are, for example, octane, toluene and/or xylenes (o-xylene, m-xylene, p-xylene).

The tertiary amine which is preferably to be used in the process of the invention is an amine of the general formula

NR¹R²R³ (A1)

in which the radicals R¹, R², R³ are identical or different and are each, independently of one another, an unbranched or branched, acyclic or cyclic, aliphatic, araliphatic or aromatic radical having in each case from 1 to 16 carbon atoms, preferably from 1 to 12 carbon atoms, where individual carbon atoms can also be, independently of one another, replaced by a heterogroup selected from among the groups —O— and >N— and two or three radicals can also be joined to one another to form a chain comprising at least four atoms. In a particularly preferred embodiment, a tertiary amine of the general formula (A1) is used, with the proviso that the total number of carbon atoms is at least 9.

As suitable tertiary amines (A1), mention may be made by way of example of:
tri-n-propylamine, tri-n-butylamine, tri-n-pentylamine, tri-n-hexylamine, tri-n-heptylamine, tri-n-octylamine, tri-n-nonylamine, tri-n-decylamine, tri-n-undecylamine, tri-n-dodecylamine, tri-n-tridecylamine, tri-n-tetradecylamine, tri-n-pentadecylamine, tri-n-hexadecylamine, tri(2-ethylhexyl)amine.
dimethyldecylamine, dimethyldodecylamine, dimethyltetradecylamine, ethyldi(2-propyl)amine, dioctylmethylamine, dihexylmethylamine.
tricyclopentylamine, tricyclohexylamine, tricycloheptylamine, tricyclooctylamine and derivatives thereof substituted by one or more methyl, ethyl, 1-propyl, 2-propyl, 1-butyl, 2-butyl or 2-methyl-2-propyl groups.
dimethylcyclohexylamine, methyldicyclohexylamine, diethylcyclohexylamine, ethyldicyclohexylamine, dimethylcyclopentylamine, methyldicyclopentylamine.
triphenylamine, methyldiphenylamine, ethyldiphenylamine, propyldiphenylamine, butyldiphenylamine, 2-ethylhexyldiphenylamine, dimethylphenylamine, diethylphenylamine, dipropylphenylamine, dibutylphenylamine, bis(2-ethylhexyl)-phenylamine, tribenzylamine, methyldibenzylamine, ethyldibenzylamine and derivatives thereof substituted by one or more methyl, ethyl, 1-propyl, 2-propyl, 1-butyl, 2-butyl or 2-methyl-2-propyl groups.
N—C₁-C₁₂-alkylpiperidines, N,N-di-C₁-C₁₂-alkylpiperazines, N—C₁-C₁₂-alkylpyrrolidones, N—C₁-C₁₂-alkylimidazoles and derivatives thereof substituted by one or more methyl, ethyl, 1-propyl, 2-propyl, 1-butyl, 2-butyl or 2-methyl-2-propyl groups.
1,8-diazabicyclo[5.4.0]undec-7-ene (“DBU”), 1,4-diazabicyclo[2.2.2]octane (“DABCO”), N-methyl-8-azabicyclo[3.2.1]octane (“tropane”), N-methyl-9-azabicyclo[3.3.1]nonane (“granatane”), 1-azabicyclo[2.2.2]octane (“quinuclidine”).

Mixtures of two or more different tertiary amines (A1) can also be used in the process of the invention.

Particular preference is given to using an amine in which the radicals R¹, R², R³ are selected independently from the group consisting of C₁-C₁₂-alkyl, C₅-C₈-cycloalkyl, benzyl and phenyl as tertiary amine (A1) in the process of the invention.

Particular preference is given to using a saturated amine, i.e. an amine comprising only single bonds, as tertiary amine (A1) in the process of the invention.

Very particular preference is given to using an amine of the general formula (A1) in which the radicals R¹, R², R³ are selected independently from the group consisting of C₅-C₈-alkyl, in particular tri-n-pentylamine, tri-n-hexylamine, tri-n-heptylamine, tri-n-octylamine, dimethylcyclohexylamine, methyldicyclohexylamine, dioctylmethylamine and dimethyldecylamine, as tertiary amine in the process of the invention.

In an embodiment of the process of the invention, one tertiary amine of the general formula (A1) is used.

In particular, an amine of the general formula (A1) in which the radicals R¹, R², R³ are selected independently from among C₅- and C₆-alkyl is used as tertiary amine. Tri-n-hexylamine is most preferably used as tertiary amine of the general formula (A1) in the process of the invention.

The tertiary amine (A1) is preferably present in liquid form in all process steps in the process of the invention. However, this is not an absolute requirement. It would also be sufficient if the tertiary amine (A1) were to be at least dissolved in suitable solvents. Suitable solvents are in principle those which are chemically inert in respect of the hydrogenation of carbon dioxide, in which the tertiary amine (A1) and the catalyst dissolve readily and in which, conversely, the polar solvent and the formic acid-amine adduct (A2) are sparingly soluble. Possibilities are therefore in principle chemically inert, nonpolar solvents such as aliphatic, aromatic or araliphatic hydrocarbons, for example octane and higher alkanes, toluene, xylenes.

In the process of the invention, at least one polar solvent selected from the group consisting of methanol, ethanol, 1-propanol, 2-propanol, 1-butanol, 2-butanol, 2-methyl-1-propanol and water is used in the hydrogenation of carbon dioxide in process step (a).

For the purposes of the present invention, the term “polar solvent” refers both to one polar solvent and also mixtures of two or more polar solvents.

The polar solvent used in the process of the invention is preferably selected or matched to the tertiary amine (A1) in such a way that the phase behavior in the hydrogenation reactor in process step (a) preferably satisfies the following criteria: the polar solvent should preferably be selected so that the hydrogenation mixture (H), optionally after addition of water, obtained in process step (a) is an at least two-phase mixture. The polar solvent should be enriched in the lower phase (L1), i.e. the lower phase (L1) should comprise the major part of the polar solvent. For the purposes of the present invention, “enriched” or “major part” in the context of the polar solvent means a proportion by weight of the polar solvent in the lower phase (L1) of >50% based on the total weight of the polar solvent in the liquid phases (upper phase (U1) and lower phase (L1)) in the hydrogenation reactor.

The proportion by weight of the polar solvent in the lower phase (L1) is preferably >70%, in particular >90%, in each case based on the total weight of the polar solvent in the upper phase (U1) and the lower phase (L1).

The choice of the polar solvent which satisfies the abovementioned criteria is generally made by means of a simple experiment in which the phase behavior and solubility of the polar solvent in the liquid phases (upper phase (U1) and lower phase (L1)) are determined experimentally under the process conditions in process step (a).

The polar solvent can be a pure polar solvent or a mixture of two or more polar solvents, as long as the polar solvent or mixture of polar solvents satisfies the abovementioned criteria in respect of phase behavior and solubility in the upper phase (U1) and the lower phase (L1) in the hydrogenation reactor in process step (a).

In an embodiment of the process of the invention, a single-phase hydrogenation mixture is firstly obtained in step (a) and this is converted by addition of water into the two-phase hydrogenation mixture (H).

In a further embodiment of the process of the invention, the two-phase hydrogenation mixture (H) is obtained directly in step (a). The two-phase hydrogenation mixture (H) obtained according to this embodiment can be passed directly to the work-up according to step (b). It is also possible for water to be additionally added to the two-phase hydrogenation mixture (H) before the further work-up in step (b). This can lead to an increase in the partition coefficient P_{K}.

When a mixture of alcohol (selected from the group consisting of methanol, ethanol, 1-propanol, 2-propanol, 1-butanol, 2-butanol and 2-methyl-1-propanol) and water is used as polar solvent, the ratio of alcohol to water is selected so that, together with the formic acid-amine adduct (A2) and the tertiary amine (A1), an at least two phase hydrogenation mixture (H) comprising the upper phase (U1) and the lower phase (L1) is formed.

It is also possible, for the case where a mixture of alcohol (selected from the group consisting of methanol, ethanol, 1-propanol, 2-propanol, 1-butanol, 2-butanol and 2-methyl-1-propanol) and water is used as polar solvent, that the ratio of alcohol to water is selected so that, together with the formic acid-amine adduct (A2) and the tertiary amine (A1), a single-phase hydrogenation mixture is initially formed and is subsequently converted into the two-phase hydrogenation mixture (H) by addition of water.

In a further particularly preferred embodiment, water, methanol or a mixture of water and methanol is used as polar solvent.

The use of diols and formic esters thereof, polyols and formic esters thereof, sulfones, sulfoxides and open-chain or cyclic amides as polar solvent is not preferred. In a preferred embodiment, these polar solvents are not present in the reaction mixture (Rg).

The molar ratio of the polar solvent or solvent mixture used in process step (a) of the process of the invention to the tertiary amine (A1) used is generally from 0.5 to 30 and preferably from 1 to 20.

The complex catalyst used in process step (a) of the process of the invention for hydrogenating carbon dioxide comprises at least one element selected from groups 8, 9 and 10 of the Periodic Table (nomenclature according to IUPAC). Groups 8, 9 and 10 of the Periodic Table comprise Fe, Co, Ni, Ru, Rh, Pd, Os, Ir and Pt. In process step (a), it is possible to use one complex catalyst or a mixture of two or more complex catalysts. Preference is given to using one complex catalyst. For the purposes of the present invention, the term “complex catalyst” refers to both one complex catalyst and mixtures of two or more complex catalysts.

The complex catalyst preferably comprises at least one element selected from the group consisting of Ru, Rh, Pd, Os, Ir and Pt, particularly preferably at least one element from the group consisting of Ru, Rh and Pd. The complex catalyst very particularly preferably comprises Ru.

Preference is given to using a complex-type compound of the above-mentioned elements as complex catalyst. The reaction in process step (a) is preferably carried out homogeneously catalyzed.

For the purposes of the present invention, homogeneously catalyzed means that the catalytically active part of the complex catalyst is at least partly present in solution in the liquid reaction medium. In a preferred embodiment, at least 90% of the complex catalyst used in process step (a) is present in solution in the liquid reaction medium, more preferably at least 95%, particularly preferably more than 99%, and the complex catalyst is most preferably entirely present in solution in the liquid reaction medium (100%), in each case based on the total amount of the complex catalyst present in the liquid reaction medium.

The amount of the metal components of the complex catalyst in process step (a) is generally from 0.1 to 5000 ppm by weight, preferably from 1 to 800 ppm by weight and particularly preferably from 5 to 800 ppm by weight, in each case based on the total liquid reaction mixture (Rg) in the hydrogenation reactor. The complex catalyst is selected so that it is enriched in the upper phase (U1), i.e. the upper phase (U1) comprises the major part of the complex catalyst. For the purposes of the present invention, “enriched” or “major part” in the context of the complex catalyst means a partition coefficient of the complex catalyst P_{K}=[concentration of the complex catalyst in the upper phase (U1)]/[concentration of the complex catalyst in the lower phase (L1)] of >1. Preference is given to a partition coefficient P_{K}>1.5 and particular preference is given to a partition coefficient P_{K}>4. The choice of the complex catalyst is generally made by means of a simple experiment in which the phase behavior and the solubility of the complex catalyst in the liquid phases (upper phase (U1) and lower phase (L1)) are determined experimentally under the process conditions in process step (a).

Owing to their good solubility in tertiary amines, homogeneous complex catalysts, in particular a metal-organic complex comprising an element of group 8, 9 or 10 of the Periodic Table and at least one phosphine group having at least one unbranched or branched, acyclic or cyclic, aliphatic radical having from 1 to 12 carbon atoms, where individual carbon atoms may also be substituted by >P—, are preferably used as complex catalysts in the process of the invention. The term “branched cyclic aliphatic radicals” also encompasses radicals such as —CH₂—C₆H₁₁. Suitable radicals are, for example, methyl, ethyl, 1-propyl, 2-propyl, 1-butyl, 1-(2-methyl)propyl, 2-(2-methyl)propyl, 1-pentyl, 1-hexyl, 1-heptyl, 1-octyl, 1-nonyl, 1-decyl, 1-undecyl, 1-dodecyl, cyclopentyl, cyclohexyl, cycloheptyl and cyclooctyl, methylcyclopentyl, methylcyclohexyl, 1-(2-methyl)pentyl, 1-(2-ethyl)hexyl, 1-(2-propyl)heptyl and norbornyl. The unbranched or branched, acyclic or cyclic, aliphatic radical preferably comprises at least 1 and preferably not more than 10 carbon atoms. In the case of an exclusively cyclic radical in the abovementioned sense, the number of carbon atoms is from 3 to 12 and preferably at least 4 and preferably not more than 8 carbon atoms. Preferred radicals are ethyl, 1-butyl, sec-butyl, 1-octyl and cyclohexyl.

The phosphine group can comprise one, two or three of the above-mentioned unbranched or branched, acyclic or cyclic, aliphatic radicals. These can be identical or different. The phosphine group preferably comprises three of the above-mentioned unbranched or branched, acyclic or cyclic, aliphatic radicals, with particular preference being given to all three radicals being identical. Preferred phosphines are P(n-CₙH₂ₙ₊₁)₃ where n is from 1 to 10, particularly preferably tri-n-butylphosphine, tri-n-octylphosphine and 1,2-bis(dicyclohexylphosphino)ethane.

As mentioned above, individual carbon atoms in the abovementioned unbranched or branched, acyclic or cyclic, aliphatic radicals can also be substituted by >P—. Polydentate, for example bidentate or tridentate, phosphine ligands are thus also comprised. These preferably comprise the groups

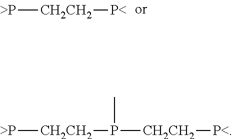

If the phosphine group comprises radicals other than the abovementioned unbranched or branched, acyclic or cyclic, aliphatic radicals, these generally correspond to those which are otherwise customarily used in phosphine ligands for metal-organic complex catalysts. Examples which may be mentioned are phenyl, tolyl and xylyl.

The metal-organic complex can comprise one or more, for example two, three or four, of the abovementioned phosphine groups having at least one unbranched or branched, acyclic or cyclic, aliphatic radical. The remaining ligands of the metal-organic complex can vary in nature. Illustrative examples which may be mentioned are hydride, fluoride, chloride, bromide, iodide, formate, acetate, propionate, carboxylate, acetylacetonate, carbonyl, DMSO, hydroxide, trialkylamine, alkoxide.

The homogeneous catalysts can be produced directly in their active form or only under reaction conditions from conventional standard complexes such as [M(p-cymene)Cl₂]₂, [M(benzene)Cl₂]ₙ, [M(COD)(allyl)], [MCl₃×H₂O], [M(acetylacetonate)₃], [M(COD)Cl₂]₂, [M(DMSO)₄Cl₂] where M is an element of group 8, 9 or 10 of the Periodic Table with addition of the appropriate phosphine ligand(s).

Homogeneous complex catalysts which are preferred in the process of the invention are selected from the group consisting of [Ru(PⁿBu₃)₄(H)₂], [Ru(Pⁿoctyl₃)₄(H)₂], [Ru(PⁿBu₃)₂(1,2-bis(dicyclohexylphosphino)ethane)(H)₂], [Ru(Pⁿoctyl₃)₂(1,2-bis(dicyclohexylphosphino)ethane)(H)₂], [Ru(PEt₃)₄(H)₂], [Ru(Pⁿoctyl₃)(1,2-bis(dicyclohexylphosphino)ethane)(CO)(H)₂], [Ru(Pⁿoctyl₃)(1,2-bis(dicyclohexylphosphino)ethane)(CO)(H)(HCOO)], [Ru(Pⁿbutyl₃)(1,2-bis(dicyclohexylphosphino)ethane)(CO)(H)₂], [Ru(Pⁿbutyl₃)(1,2-bis(dicyclohexylphosphino)ethane)(CO)(H)(HCOO)], [Ru(Pⁿethyl₃)(1,2-bis(dicyclohexylphosphino)ethane)(CO)(H)₂], [Ru(Pⁿethyl₃)(1,2-bis(dicyclohexylphosphino)ethane)(CO)(H)(HCOO)], [Ru(Pⁿoctyl₃)(1,1-bis(dicyclohexylphosphino)methane)(CO)(H)₂], [Ru(Pⁿoctyl₃)(1,1-bis(dicyclohexylphosphino)methane)(CO)(H)(HCOO)], [Ru(Pⁿbutyl₃)(1,1-bis(dicyclohexylphosphino)methane)(CO)(H)₂], [Ru(Pⁿbutyl₃)(1,1-bis(dicyclohexylphosphino)methane)(CO)(H)(HCOO)], [Ru(Pⁿethyl₃)(1,1-bis(dicyclohexylphosphino)methane)(CO)(H)₂], [Ru(Pⁿethyl₃)(1,1-bis(dicyclohexylphosphino)methane)(CO)(H)(HCOO)], [Ru(Pⁿoctyl₃)(1,2-bis(diphenylphosphino)ethane)(CO)(H)₂], [Ru(Pⁿoctyl₃)(1,2-bis(diphenylphosphino)ethane)(CO)(H)(HCOO)], [Ru(Pⁿbutyl₃)(1,2-bis(diphenylphosphino)ethane)(CO)(H)₂], [Ru(Pⁿbutyl₃)(1,2-bis(diphenylphosphino)ethane)(CO)(H)(HCOO)], [Ru(Pⁿethyl₃)(1,2-bis(diphenylphosphino)ethane)(CO)(H)₂], [Ru(Pⁿethyl₃)(1,2-bis(diphenylphosphino)ethane)(CO)(H)(HCOO)], [Ru(Pⁿoctyl₃)(1,1-bis(diphenylphosphino)methane)(CO)(H)₂], [Ru(Pⁿoctyl₃)(1,1-bis(diphenylphosphino)methane)(CO)(H)(HCOO)], [Ru(Pⁿbutyl₃)(1,1-bis(diphenylphosphino)methane)(CO)(H)₂], [Ru(Pⁿbutyl₃)(1,1-bis(diphenylphosphino)methane)(CO)(H)(HCOO)], [Ru(Pⁿethyl₃)(1,1-bis(diphenylphosphino)methane)(CO)(H)₂], [Ru(Pⁿethyl₃)(1,1-bis(diphenylphosphino)methane)(CO)(H)(HCOO)].

TOF (turnover frequency) values of greater than 1000 h⁻¹ can be achieved in the hydrogenation of carbon dioxide by means of these catalysts.

The hydrogenation of carbon dioxide in process step (a) occurs in the liquid phase, preferably at a temperature in the range from 20 to 200° C. and a total pressure in the range from 0.2 to 30 MPa abs. The temperature is preferably at least 30° C. and particularly preferably at least 40° C. and preferably not more than 150° C., particularly preferably not more than 120° C. and very particularly preferably not more than 80° C. The total pressure is preferably at least 1 MPa abs and particularly preferably at least 5 MPa abs and preferably not more than 20 MPa abs.

In a preferred embodiment, the hydrogenation in process step (a) is carried out at a temperature in the range from 40 to 80° C. and a pressure in the range from 5 to 20 MPa abs.

The partial pressure of carbon dioxide in process step (a) is generally at least 0.5 MPa and preferably at least 2 MPa and generally not more than 8 MPa. In a preferred embodiment, the hydrogenation in process step (a) is carried out at a partial pressure of carbon dioxide in the range from 2 to 7.3 MPa.

The partial pressure of hydrogen in process step (a) is generally at least 0.5 MPa and preferably at least 1 MPa and generally not more than 25 MPa and preferably not more than 15 MPa. In a preferred embodiment, the hydrogenation in process step (a) is carried out at a partial pressure of hydrogen in the range from 1 to 15 MPa.

The molar ratio of hydrogen to carbon dioxide in the reaction mixture (Rg) in the hydrogenation reactor is preferably from 0.1 to 10 and particularly preferably from 1 to 3.

The molar ratio of carbon dioxide to tertiary amine (A1) in the reaction mixture (Rg) in the hydrogenation reactor is preferably from 0.1 to 10 and particularly preferably from 0.5 to 3.

As hydrogenation reactors, it is in principle possible to use all reactors which are suitable for gas/liquid reactions at the given temperature and the given pressure. Suitable standard reactors for gas-liquid reaction systems are described, for example, in K. D. Henkel, “Reactor Types and Their Industrial Applications”, in Ullmann's Encyclopedia of Industrial Chemistry, 2005, Wiley-VCH Verlag GmbH & Co. KGaA, DOI: 10.1002/14356007.b04_{—}087, chapter 3.3 “Reactors for gas-liquid reactions”. Examples which may be mentioned are stirred tank reactors, tube reactors and bubble column reactors.

The hydrogenation of carbon dioxide can be carried out batchwise or continuously in the process of the invention. In the case of a batch process, the reactor is charged with the desired liquid and optionally solid starting materials and auxiliaries, after which carbon dioxide and the polar solvent are injected to the desired pressure at the desired temperature. After the reaction is complete, the reactor is generally depressurized and the two liquid phases formed are separated from one another. In a continuous process, the starting materials and auxiliaries, including the carbon dioxide and hydrogen, are introduced continuously. Accordingly, the liquid phases are discharged continuously from the reactor so that the liquid level in the reactor remains, on average, constant. Preference is given to the continuous hydrogenation of carbon dioxide.

The average residence time of the components comprised in the reaction mixture (Rg) in the hydrogenation reactor is generally from 5 minutes to 5 hours.

The homogeneously catalyzed hydrogenation in process step (a) gives a hydrogenation mixture (H) which comprises the complex catalyst, the polar solvent, the tertiary amine (A1) and the at least one formic acid-amine adduct (A2).

For the purposes of the present invention, the term “formic acid-amine adduct (A2)” refers to both one formic acid-amine adduct (A2) and mixtures of two or more formic acid-amine adducts (A2). Mixtures of two or more formic acid-amine adducts (A2) are obtained in process step (a) when two or more tertiary amines (A1) are used in the reaction mixture (Rg).

In a preferred embodiment of the process of the invention, a reaction mixture (Rg) comprising one tertiary amine (A1) is used in process step (a) to give a hydrogenation mixture (H) comprising one formic acid-amine adduct (A2).

In a particularly preferred embodiment of the process of the invention, a reaction mixture (Rg) comprising tri-n-hexylamine as tertiary amine (A1) is used in process step (a) to give a hydrogenation mixture (H) comprising the formic acid-amine adduct of tri-n-hexylamine and formic acid, corresponding to the formula (A3) below

N(n-hexyl)₃*xᵢHCOOH (A3).

In the formic acid-amine adduct of the general formula (A2) or (A3), the radicals R¹, R², R³ have the meanings given above for the tertiary amine of the formula (A1), with the preferences indicated there applying analogously.

In the general formulae (A2) and (A3), xᵢ is in the range from 0.4 to 5. The factor xᵢ indicates the average composition of the formic acid-amine adduct (A2) or (A3), i.e. the ratio of bound tertiary amine (A1) to bound formic acid in the formic acid-amine adduct (A2) or (A3).

The factor xᵢ can be determined, for example, by determining the formic acid content by acid-base titration with an alcoholic KOH solution against phenolphthalein. The factor xᵢ can also be determined by determining the amine content by gas chromatography. The precise composition of the formic acid-amine adduct (A2) or (A3) is dependent on many parameters such as the concentrations of formic acid and tertiary amine (A1), the pressure, the temperature and the presence and nature of further components, in particular the polar solvent.

The composition of the formic acid-amine adduct (A2) or (A3), i.e. the factor xᵢ, can therefore also alter over the individual process steps. Thus, for example, a formic acid-amine adduct (A2) or (A3) having a relatively high formic acid content is generally formed after removal of the polar solvent, with the excess bound tertiary amine (A1) being eliminated from the formic acid-amine adduct (A2) and a second phase being formed.

Process step (a) generally gives a formic acid-amine adduct (A2) or (A3) in which xᵢ is in the range from 0.4 to 5, preferably in the range from 0.7 to 1.6.

The formic acid-amine adduct (A2) is enriched in the lower phase (L1), i.e. the lower phase (L1) comprises the major part of the formic acid-amine adduct. For the purposes of the present invention, “enriched” or “major part” in the context of the formic acid-amine adduct (A2) means a proportion by weight of the formic acid-amine adduct (A2) in the lower phase (L1) of >50% based on the total weight of the formic acid-amine adduct (A2) in the liquid phases (upper phase (U1) and lower phase (L1)) in the hydrogenation reactor.

The proportion by weight of the formic acid-amine adduct (A2) in the lower phase (L1) is preferably >70%, in particular >90%, in each case based on the total weight of the formic acid-amine adduct (A2) in the upper phase (U1) and the lower phase (L1).

### Work-Up of the Hydrogenation Mixture (H); Process Step (b)

The hydrogenation mixture (H) obtained in the hydrogenation of carbon dioxide in process step (a) preferably has two liquid phases and is worked up further in process step (b) according to one of the steps (b1), (b2) or (b3).

Work-Up According to Process Step (b1)

In a preferred embodiment, the hydrogenation mixture (H) is worked up further according to step (b1). The invention therefore also provides a process for preparing formic acid, which comprises the steps
(a) homogeneously catalyzed reaction of a reaction mixture (Rg) comprising carbon dioxide, hydrogen, at least one polar solvent selected from the group consisting of methanol, ethanol, 1-propanol, 2-propanol, 1-butanol, 2-butanol, 2-methyl-1-propanol and water and also at least one tertiary amine of the general formula (A1)

NR¹R²R³ (A1),
where
R¹, R², R³ are each, independently of one another, an unbranched or branched, acyclic or cyclic, aliphatic, araliphatic or aromatic radical having in each case from 1 to 16 carbon atoms, where individual carbon atoms may, independently of one another, also be replaced by a heterogroup selected from among the groups —O— and >N— and two or all three radicals can also be joined to one another to form a chain comprising at least four atoms,
in the presence of at least one complex catalyst comprising at least one element selected from groups 8, 9 and 10 of the Periodic Table,
in a hydrogenation reactor
to give, optionally after addition of water, a two-phase hydrogenation mixture (H) comprising
an upper phase (U1), which comprises the at least one complex catalyst and the at least one tertiary amine (A1) and
a lower phase (L1) which comprises the at least one polar solvent, residues of the at least one complex catalyst and also at least one formic acid-amine adduct of the general formula (A2),

NR¹R²R³*xᵢHCOOH (A2),
where
xᵢ is in the range from 0.4 to 5 and
R¹, R², R³ are as defined above,
(b1) phase separation of the hydrogenation mixture (H) obtained in step (a) into the upper phase (U1) and the lower phase (L1) in a first phase separation apparatus
(c) separation of the at least one polar solvent from the lower phase (L1) in a first distillation unit to give
   a distillate (D1) comprising the at least one polar solvent, which is recirculated to the hydrogenation reactor in step (a), and
   a two-phase bottoms mixture (S1) comprising
   an upper phase (U2) which comprises the at least one tertiary amine (A1) and a lower phase (L2) which comprises the at least one formic acid-amine adduct (A2),
(d) optionally work-up of the first bottoms mixture (S1) obtained in step (c) by phase separation in a second phase separation apparatus to give the upper phase (U2) and the lower phase (L2),
(e) dissociation of the at least one formic acid-amine adduct (A2) comprised in the bottoms mixture (S1) or optionally in the lower phase (L2) in a thermal dissociation unit to give the at least one tertiary amine (A1), which is recirculated to the hydrogenation reactor in step (a), and formic acid, which is discharged from the thermal dissociation unit,
   wherein carbon monoxide is added to the lower phase (L1) directly before and/or during step (c)
   and/or
   carbon monoxide is added to the bottoms mixture (S1) or optionally to the lower phase (L2) directly before and/or during step (e).

Here, the hydrogenation mixture (H) obtained in process step (a) is worked up further by phase separation in a first phase separation apparatus to give a lower phase (L1) comprising the at least one formic acid-amine adduct (A2), the at least one polar solvent and residues of the at least one complex catalyst and also an upper phase (U1) comprising the at least one complex catalyst and the at least one tertiary amine (A1).

In a preferred embodiment, the upper phase (U1) is recirculated to the hydrogenation reactor. The lower phase (L1) is, in a preferred embodiment, fed to the first distillation apparatus in process step (c). Recirculation of a further liquid phase which comprises unreacted carbon dioxide and is present over the two liquid phases and also of a gas phase comprising unreacted carbon dioxide and/or unreacted hydrogen to the hydrogenation reactor may also be advantageous. It may be desirable, for example to discharge undesirable by-products or impurities, to discharge part of the upper phase (U1) and/or part of the liquid or gaseous phases comprising carbon dioxide or carbon dioxide and hydrogen from the process.

The separation of the hydrogenation mixture (H) obtained in process step (a) is generally carried out by gravimetric phase separation. Suitable phase separation apparatuses are, for example, standard apparatuses and standard methods as may be found, for example, in E. Müller et al., “Liquid-liquid Extraction”, in Ullman's Encyclopedia of Industrial Chemistry, 2005, Wiley-VCH Verlag GmbH & Co. KGaA, DOI: 10.1002/14356007.b93_{—}06, chapter 3 “Apparatus”. In general, the liquid phase enriched in the formic acid-amine adducts (A2) and the polar solvent is heavier and forms the lower phase (L1).

The phase separation can, for example, be effected by depressurization to about or close to atmospheric pressure and cooling of the liquid hydrogenation mixture, for example to about or close to ambient temperature.

In the context of the present invention, it has been found that in the present system, i.e. a lower phase (L1) enriched in the formic acid-amine adducts (A2) and the polar solvent and an upper phase (U1) enriched in the tertiary amine (A1) and the complex catalyst, the two liquid phases separate very well, even at significantly elevated pressure, when a suitable combination of the polar solvent and the tertiary amine (A1) is selected. For this reason, the polar solvent and the tertiary amine (A1) in the process of the invention are selected so that the separation of the lower phase (L1) enriched in the formic acid-amine adducts (A2) and the polar solvent from the upper phase (U1) enriched in tertiary amine (A1) and complex catalyst and also the recirculation of the upper phase (U1) to the hydrogenation reactor can be carried out at a pressure of from 1 to 30 MPa abs. Corresponding to the total pressure in the hydrogenation reactor, the pressure is preferably not more than 15 MPa abs. It is thus possible to separate the two liquid phases (upper phase (U1) and lower phase (L1)) from one another without prior depressurization in the first phase separation apparatus and to recirculate the upper phase (U1) to the hydrogenation reactor without an appreciable increase in pressure.

It is also possible to carry out the phase separation directly in the hydrogenation reactor. In this embodiment, the hydrogenation reactor simultaneously functions as the first phase separation apparatus and the process steps (a) and (b1) are both carried out in the hydrogenation reactor. Here, the upper phase (U1) remains in the hydrogenation reactor and the lower phase (L1) is fed to the first distillation apparatus in process step (c).

In one embodiment, the process of the invention is carried out with the pressure and the temperature in the hydrogenation reactor and in the first phase separation apparatus being the same or approximately the same, with approximately the same meaning, for the present purposes, a pressure difference of up to +/−0.5 MPa or a temperature difference of up to +/−10° C.

It has surprisingly also been found that in the case of the present system, the two liquid phases (upper phase (U1) and lower phase (L1)) separate from one another very readily even at an elevated temperature corresponding to the reaction temperature in the hydrogenation reactor. In this case, no cooling for the phase separation in process step (b1) and subsequent heating of the recirculated upper phase (U1) is necessary, which likewise saves energy.

Work-Up According to Process Step (b3)

In a further preferred embodiment, the hydrogenation mixture (H) is worked up further according to step (b3). The invention therefore also provides a process for preparing formic acid, which comprises the steps
(a) homogeneously catalyzed reaction of a reaction mixture (Rg) comprising carbon dioxide, hydrogen, at least one polar solvent selected from the group consisting of methanol, ethanol, 1-propanol, 2-propanol, 1-butanol, 2-butanol, 2-methyl-1-propanol and water and also at least one tertiary amine of the general formula (A1)

NR¹R²R³ (A1),
where
R¹, R², R³ are each, independently of one another, an unbranched or branched, acyclic or cyclic, aliphatic, araliphatic or aromatic radical having in each case from 1 to 16 carbon atoms, where individual carbon atoms may, independently of one another, also be replaced by a heterogroup selected from among the groups —O— and >N— and two or all three radicals can also be joined to one another to form a chain comprising at least four atoms,
in the presence of at least one complex catalyst comprising at least one element selected from groups 8, 9 and 10 of the Periodic Table,
in a hydrogenation reactor
to give, optionally after addition of water, a two-phase hydrogenation mixture (H) comprising
an upper phase (U1), which comprises the at least one complex catalyst and the at least one tertiary amine (A1) and
a lower phase (L1) which comprises the at least one polar solvent, residues of the at least one complex catalyst and also at least one formic acid-amine adduct of the general formula (A2),

NR¹R²R³*xᵢHCOOH (A2),
where
xᵢ is in the range from 0.4 to 5 and
R¹, R², R³ are as defined above,
(b3) phase separation of the hydrogenation mixture (H) obtained in step (a) into the upper phase (U1) and the lower phase (L1) in a first phase separation apparatus and extraction of the residues of the at least one complex catalyst from the lower phase (L1) by means of an extractant comprising the at least one tertiary amine (A1) in an extraction unit to give
   a raffinate (R2) comprising the at least one formic acid-amine adduct (A2) and the at least one polar solvent and
   an extract (E2) comprising the at least one tertiary amine (A1) and the residues of the at least one complex catalyst,
(c) separation of the at least one polar solvent from the raffinate (R2) in a first distillation unit to give
   a distillate (D1) comprising the at least one polar solvent, which is recirculated to the hydrogenation reactor in step (a), and
   a two-phase bottoms mixture (S1) comprising
   an upper phase (U2) which comprises the at least one tertiary amine (A1) and a lower phase (L2) which comprises the at least one formic acid-amine adduct (A2),
(d) optionally work-up of the first bottoms mixture (S1) obtained in step (c) by phase separation in a second phase separation apparatus to give the upper phase (U2) and the lower phase (L2),
(e) dissociation of the at least one formic acid-amine adduct (A2) comprised in the bottoms mixture (S1) or optionally in the lower phase (L2) in a thermal dissociation unit to give the at least one tertiary amine (A1), which is recirculated to the hydrogenation reactor in step (a), and formic acid, which is discharged from the thermal dissociation unit,
wherein carbon monoxide is added to the raffinate (R2) directly before and/or during step (c)
and/or
carbon monoxide is added to the bottoms mixture (S1) or optionally to the lower phase (L2) directly before and/or during step (e).

Here, the hydrogenation mixture (H) obtained in process step (a) is, as described above for process step (b1), separated in the first phase separation apparatus into the lower phase (L1) and the upper phase (U1) which is recirculated to the hydrogenation reactor. With regard to the phase separation in process step (b3), what has been said in respect of process step (b1), including preferences, applies analogously. In the work-up according to step (b3), too, the phase separation can be carried out directly in the hydrogenation reactor. In this embodiment, the hydrogenation reactor simultaneously functions as first phase separation apparatus. The upper phase (U1) then remains in the hydrogenation reactor and the lower phase (L1) is fed to the extraction unit.

The lower phase (L1) obtained after phase separation is subsequently subjected to an extraction with at least one tertiary amine (A1) as extractant to separate off the residues of the complex catalyst in an extraction unit to give a raffinate (R2) comprising the at least one formic acid-amine adduct (A2) and the at least one polar solvent and an extract (E2) comprising the at least one tertiary amine (A1) and the residues of the complex catalyst.

In a preferred embodiment, the same tertiary amine (A1) comprised in the reaction mixture (Rg) in process step (a) is used as extractant, so that what has been said in respect of process step (a), including preferences, in respect of the tertiary amine (A1) applies analogously to process step (b3).

The extract (E2) obtained in process step (b3) is, in a preferred embodiment, recirculated to the hydrogenation reactor in process step (a). This makes efficient recovery of the expensive complex catalyst possible. The raffinate (R2) is, in a preferred embodiment, fed to the first distillation apparatus in process step (c).

The tertiary amine (A1) which is obtained in the thermal dissociation unit in process step (e) is preferably used as extractant in process step (b3). In a preferred embodiment, the tertiary amine (A1) obtained in the thermal dissociation unit in process step (e) is recirculated to the extraction unit in process step (b3).

The extraction in process step (b3) is generally carried out at temperatures in the range from 30 to 100° C. and pressures in the range from 0.1 to 8 MPa. The extraction can also be carried out under hydrogen pressure.

The extraction of the complex catalyst can be carried out in any suitable apparatus known to those skilled in the art, preferably in countercurrent extraction columns, mixer-settler cascades or combinations of mixer-settler cascades and countercurrent extraction columns.

Optionally, not only the complex catalyst but also proportions of individual components of the polar solvent from the lower phase (L1) to be extracted are dissolved in the extractant, viz. the tertiary amine (A1). This is not a disadvantage for the process since the amount of polar solvent which has been extracted does not have to be fed to solvent removal and thus may save vaporization energy in some circumstances.

Work-Up According to Process Step (b2)

In a further preferred embodiment, the hydrogenation mixture (H) is worked up further according to step (b2). The invention therefore also provides a process for preparing formic acid, which comprises the steps
(a) homogeneously catalyzed reaction of a reaction mixture (Rg) comprising carbon dioxide, hydrogen, at least one polar solvent selected from the group consisting of methanol, ethanol, 1-propanol, 2-propanol, 1-butanol, 2-butanol, 2-methyl-1-propanol and water and also at least one tertiary amine of the general formula (A1)

NR¹R²R³ (A1),
where
R¹, R², R³ are each, independently of one another, an unbranched or branched, acyclic or cyclic, aliphatic, araliphatic or aromatic radical having in each case from 1 to 16 carbon atoms, where individual carbon atoms may, independently of one another, also be replaced by a heterogroup selected from among the groups —O— and >N— and two or all three radicals can also be joined to one another to form a chain comprising at least four atoms,
in the presence of at least one complex catalyst comprising at least one element selected from groups 8, 9 and 10 of the Periodic Table,
in a hydrogenation reactor
to give, optionally after addition of water, a two-phase hydrogenation mixture (H) comprising
an upper phase (U1), which comprises the at least one complex catalyst and the at least one tertiary amine (A1) and
a lower phase (L1) which comprises the at least one polar solvent, residues of the at least one catalyst and also at least one formic acid-amine adduct of the general formula (A2),

NR¹R²R³*xᵢHCOOH (A2),
where
xᵢ is in the range from 0.4 to 5 and
R¹, R², R³ are as defined above,
(b2) extraction of the at least one complex catalyst from the hydrogenation mixture (H) obtained in step (a) by means of an extractant comprising the at least one tertiary amine (A1) in an extraction unit to give
a raffinate (R1) comprising the at least one formic acid-amine adduct (A2) and the at least one polar solvent and
an extract (E1) comprising the at least one tertiary amine (A1) and the at least one complex catalyst
(c) separation of the at least one polar solvent from the raffinate (R1) in a first distillation unit to give
   a distillate (D1) comprising the at least one polar solvent, which is recirculated to the hydrogenation reactor in step (a), and
   a two-phase bottoms mixture (S1) comprising
   an upper phase (U2) which comprises the at least one tertiary amine (A1) and a lower phase (L2) which comprises the at least one formic acid-amine adduct (A2),
(d) optionally work-up of the bottoms mixture (S1) obtained in step (c) by phase separation in a second phase separation apparatus to give the upper phase (U2) and the lower phase (L2),
(e) dissociation of the at least one formic acid-amine adduct (A2) comprised in the bottoms mixture (S1) or optionally in the lower phase (L2) in a thermal dissociation unit to give the at least one tertiary amine (A1), which is recirculated to the hydrogenation reactor in step (a), and formic acid, which is discharged from the thermal dissociation unit,
wherein carbon monoxide is added to the raffinate (R1) directly before and/or during step (c)
and/or
carbon monoxide is added to the bottoms mixture (S1) or optionally to the lower phase (L2) directly before and/or during step (e).

Here, the hydrogenation mixture (H) obtained in process step (a) is fed directly, without prior phase separation, to the extraction unit. What has been said above in respect of the extraction for process step (b3), including preferences, applies analogously to process step (b2).

The hydrogenation mixture (H) is subjected to an extraction with at least one tertiary amine (A1) as extractant to separate off the catalyst in an extraction unit to give a raffinate (R1) comprising the at least one formic acid-amine adduct (A2) and the at least one polar solvent and an extract (E1) comprising the at least one tertiary amine (A1) and the at least one complex catalyst.

In a preferred embodiment, the same tertiary amine (A1) comprised in the reaction mixture (Rg) in process step (a) is used as extractant, so that what has been said above in respect of the tertiary amine (A1) for process step (a), including preferences, applies analogously to process step (b2).

The extract (E1) obtained in process step (b2) is, in a preferred embodiment, recirculated to the hydrogenation reactor in process step (a). This makes efficient recovery of the expensive complex catalyst possible. The raffinate (R1) is, in a preferred embodiment, fed to the first distillation apparatus in process step (c).

The tertiary amine (A1) obtained in the thermal dissociation unit in process step (e) is preferably used as extractant in process step (b2). In a preferred embodiment, the tertiary amine (A1) obtained in the thermal dissociation unit in process step (e) is recirculated to the extraction unit in process step (b2).

The extraction in process step (b2) is generally carried out at temperatures in the range from 30 to 100° C. and pressures in the range from 0.1 to 8 MPa. The extraction can also be carried out under hydrogen pressure.

The extraction of the complex catalyst can be carried out in any suitable apparatus known to those skilled in the art, preferably in countercurrent extraction columns, mixer-settler cascades or combinations of mixer-settler cascades and countercurrent extraction columns.

Optionally, not only the complex catalyst but also proportions of individual components of the polar solvent from the hydrogenation mixture (H) to be extracted are dissolved in the extractant, viz. the tertiary amine (A1). This is not a disadvantage for the process since the amount of polar solvent which has been extracted does not have to be fed to solvent removal and thus may save vaporization energy in some circumstances.

### Inhibition of Traces of the Catalyst

The inhibition of the complex catalyst by means of carbon monoxide can be carried out directly before and/or during step (c) and/or directly before and/or during step (e).

In an embodiment, the inhibition is carried out exclusively directly before and/or during step (c).

In an embodiment, the inhibition is carried out exclusively directly before and/or during step (e).

In a further embodiment, the addition of carbon monoxide is carried out both directly before and/or during step (c) and directly before and/or during step (e).

### Inhibition, Step (c)

Carbon monoxide is added as inhibitor (decomposition inhibitor) to the lower phase (L1) obtained according to process step (b1), the raffinate (R1) obtained according to process step (b2) or the raffinate (R2) obtained according to process step (b3) immediately before and/or during step (c).

Although the inventive work-up of the hydrogenation mixture (H) makes effective isolation and recirculation of the complex catalyst to the hydrogenation reactor in step (a) possible, residues of the complex catalyst are still comprised in the lower phase (L1) in the work-up according to process step (b1). In the work-up according to process step (b2), the raffinate (R1) still comprises traces of the complex catalyst. In the case of the work-up according to process step (b3), too, the raffinate (R2) still comprises traces of the complex catalyst.

The lower phase (L1) obtained according to process step (b1) comprises residues of the complex catalyst in amounts of <100 ppm, preferably <80 ppm and in particular <60 ppm, in each case based on the total weight of the lower phase (L1).

The raffinate (R1) obtained according to process step (b2) comprises traces of the complex catalyst in amounts of <30 ppm, preferably <20 ppm and in particular <10 ppm, in each case based on the total weight of the raffinate (R1).

The raffinate (R2) obtained according to process step (b3) comprises traces of the complex catalyst in amounts of <30 ppm, preferably <20 ppm and in particular <10 ppm, in each case based on the total weight of the raffinate (R2).

The residues or traces of the complex catalyst in the lower phase (L1), the raffinate (R1) or the raffinate (R2) lead to redissociation of the formic acid-amine adduct (A2) into tertiary amine (A1), carbon dioxide and hydrogen in the further work-up. The redissociation of free formic acid, which may be comprised in the lower phase (L1), the raffinate (R1) or the raffinate (R2) or is formed from the formic acid-amine adduct (A2) in the further work-up, is also catalyzed by the residues or traces of the complex catalyst. The formic acid is in the case dissociated into carbon dioxide and hydrogen.

To prevent or minimize this redissociation, carbon monoxide is added as inhibitor directly before and/or during step (c).

In an embodiment of the present invention, the inhibitor is added either directly before or during step (c). In a further embodiment of the present invention, the at least one inhibitor is added directly before and during step (c). In a further embodiment, the at least one inhibitor is added only directly before step (c). In a further embodiment, the inhibitor is added only during step (c).

For the purposes of the present invention, “directly before step (c)” refers to an addition of the inhibitor to the feed to the first distillation apparatus or introduction directly into the first distillation apparatus. The addition of the inhibitor can be carried out continuously or discontinuously.

The inhibitor converts the complex catalyst into an inactive form (inhibited complex catalyst). In the inhibition, at least one ligand of the complex catalyst is replaced by carbon monoxide. Part of the ligands originally comprised in the active complex catalyst is eliminated here. The eliminated ligands are present in their free, i.e. not bound to the metal component of the complex catalyst, form (free ligands) after the inhibition. This prevents the dissociation of the formic acid-amine adduct (A2) or of the free formic acid since in the presence of carbon monoxide the complex catalyst (in its inhibited form) can no longer catalyze the redissociation of the formic acid-amine adduct (A2) or of the free formic acid.

In the absence of carbon monoxide, this reaction can be reversed again in the presence of the free ligands, thus effecting regeneration of the inhibited complex catalyst. Here, carbon monoxide is eliminated from the inhibited complex catalyst and replaced by the free ligands, forming the active complex catalyst. The regeneration can be carried out directly in the hydrogenation in step (a) if the inhibited catalyst is recirculated via the free amine (upper phase (U3)) again to the hydrogenation. It is also possible to accelerate the regeneration in a preceding step by means of thermal treatment of the inhibited catalyst.

It is possible to use carbon monoxide-comprising gases as inhibitors. In a preferred embodiment, pure carbon monoxide having a content of 99% by weight, preferably 99.5% by weight, in particular 99.9% by weight, is used, in each case based on the total weight of the gas stream used as inhibitor. It is also possible to use mixtures of carbon monoxide and hydrogen (known as synthesis gas or oxo gas) since this is often more readily available than pure carbon monoxide. The carbon monoxide content thereof is preferably from 10 to 90% by weight. In a further embodiment, the carbon monoxide can also be circulated as a cycle stream by reusing the carbon monoxide-comprising offgas from the distillation unit of the thermal dissociation unit for the inhibition. The gas stream used as inhibitor preferably consists of carbon monoxide.

The inhibitor is used in a molar ratio of from 0.5 to 1000, preferably from 1 to 30, based on the catalytically active metal component of the complex catalyst in the first distillation apparatus and/or the thermal dissociation unit.

In step (c), the inhibited complex catalyst and the free ligands are preferably present in enriched form in the upper phase (U2). The upper phase then comprises the tertiary amine (A1) and the inhibited complex catalyst and also the free ligands. The carbon monoxide which is not bound to the metal component of the inhibited complex catalyst (free carbon monoxide) is discharged from the first distillation apparatus and can be reused for inhibition of the catalyst. The embodiments and preferences for step (e) in respect of the inhibited complex catalyst apply analogously to step (c).

### Inhibition in Step (e)

In a further embodiment of the present invention, the inhibitor is added either directly before or during step (e). In a further embodiment of the present invention, the at least one inhibitor is added directly before and during step (e). In a further embodiment, the at least one inhibitor is added only directly before step (e). In a further embodiment, the inhibitor is added only during step (e).

For the purposes of the present invention, “directly before step (e)” refers to an addition of the inhibitor to the feed to the thermal dissociation unit or directly into the thermal dissociation unit. The addition of the inhibitor can be carried out continuously or discontinuously.

In step (e), the inhibited complex catalyst is, in a preferred embodiment, enriched in the upper phase (U3). The upper phase (U3) then comprises the tertiary amine (A1) and the inhibited complex catalyst and also the free ligands of the complex catalyst. The carbon monoxide-inhibited complex catalyst can be recirculated via the upper phase (U3) from the thermal dissociation to the hydrogenation in step (a). Here, the free ligands of the complex catalyst used according to the invention are selected so that they are preferentially present together with the inhibited complex catalyst in enriched form in the upper phase (U3).

In the context of the inhibited complex catalyst, “enriched” means, in respect of the process step (e), a partition coefficient

P_{ICC(e)}=[concentration of inhibited complex catalyst in the upper phase (U3)]/[concentration of inhibited complex catalyst in the lower phase (L3)]

of >1. The partition coefficient P_{ICC(e)} is preferably ≧2 and particularly preferably ≧5.

In the context of the free ligands, “enriched” in respect of the process step (e) means a partition coefficient

P_{FL(e)}=[concentration of free ligands in the upper phase (U3)]/[concentration of free ligands in the lower phase (L3)]

of >1. The partition coefficient P_{FL(e)} is i preferably 2 and particularly preferably 5.

This makes recirculation of the upper phase (U3) to the hydrogenation reactor possible without considerable amounts of the complex catalyst being lost.

The carbon monoxide which is not bound to the metal component of the inhibited complex catalyst (free carbon monoxide) is discharged from the thermal dissociation unit and can be reused for inhibition of the catalyst.

The inhibited complex catalyst can be reconverted into its active form in the absence of carbon monoxide (reactivation). It is presumed that here the carbon monoxide bound to the metal component of the inhibited complex catalyst is eliminated and replaced by free ligands. The reactivation of the inhibited complex catalyst can, in one embodiment, be carried out in process step (a). Here, the upper phase (U3) is recycled from the thermal dissociation unit to process step (a).

In a preferred embodiment, the inhibited complex catalyst comprised in upper phase (U3) is reactivated before recirculation to step (a). For this purpose, the inhibited complex catalyst is subjected to a thermal treatment in the absence of free carbon monoxide in order to convert the inhibited complex catalyst into the active form before recirculation to step (a) and thereby increase the space-time yield in the hydrogenation. In the thermal treatment, the upper phase (U3) is heated to from 100 to 200° C. under a pressure of from 10 mbar to 10 bar.

### Removal of the Polar Solvent; Process Step (c)

In process step (c), the polar solvent is separated off from the lower phase (L1), from the raffinate (R1) or from the raffinate (R2) in a first distillation apparatus. A distillate (D1) and a two-phase bottoms mixture (S1) are obtained from the first distillation apparatus. The distillate (D1) comprises the polar solvent which has been separated off and is recirculated to the hydrogenation reactor in step (a). The bottoms mixture (S1) comprises the upper phase (U2), which comprises the tertiary amine (A1), and a lower phase (L2), which comprises the formic acid-amine adduct (A2). In an embodiment of the process of the invention, the polar solvent is partly separated off in the first distillation apparatus in process step (c) so that the bottoms mixture (S1) still comprises polar solvent which has not yet been separated off. In process step (c), it is possible to separate off, for example, from 5 to 98% by weight of the polar solvent comprised in the lower phase (L1), in the raffinate (R1) or in the raffinate (R2), with preference being given to from 50 to 98% by weight, more preferably from 80 to 98% by weight and particularly preferably from 80 to 90% by weight, being separated off, in each case based on the total weight of the polar solvent comprised in the lower phase (L1), in the raffinate (R1) or in the raffinate (R2). The carbon monoxide serving as decomposition inhibitor can here either be added to the feed or introduced directly in gaseous form into the first distillation apparatus.

In a further embodiment of the process of the invention, the polar solvent is completely separated off in the first distillation apparatus in process step (c). For the purposes of the present invention, “completely separated off” means a removal of more than 98% by weight of the polar solvent comprised in the lower phase (L1), in the raffinate (R1) or in the raffinate (R2), preferably more than 98.5% by weight, particularly preferably more than 99% by weight, in particular more than 99.5% by weight, in each case based on the total weight of the polar solvent comprised in the lower phase (L1), in the raffinate (R1) or in the raffinate (R2).

The distillate (D1) which has been separated off in the first distillation apparatus is, in a preferred embodiment, recirculated to the hydrogenation reactor in step (a). When a mixture of one or more alcohols and water is used as polar solvent, it is also possible to take off a low-water distillate (D1_{wa}) and a water-rich distillate (D1_{wr}) from the first distillation apparatus. The water-rich distillate (D1_{wr}) comprises more than 50% by weight of the water comprised in the distillate (D1), preferably more than 70% by weight, particularly preferably more than 80% by weight, in particular more than 90% by weight. The low-water distillate (D1_{wa}) comprises less than 50% by weight of the water comprised in the distillate D1, preferably less than 30% by weight, particularly preferably less than 20% by weight, in particular less than 10% by weight.

In a particularly preferred embodiment, the low-water distillate (D1_{wa}) is recirculated to the hydrogenation reactor in step (a). The water-rich distillate (D1_{wr}) is added to the upper phase (U1).

The separation of the polar solvent from the lower phase (L1), the raffinate (R1) or the raffinate (R2) can, for example, be carried out in an evaporator or in a distillation unit comprising a vaporizer and column, with the column being provided with ordered packing, random packing elements and/or trays.

The at least partial removal of the polar solvent is preferably carried out at a temperature at the bottom at which no free formic acid is formed from the formic acid-amine adduct (A2) at the given pressure. The factor xᵢ of the formic acid-amine adduct (A2) in the first distillation apparatus is generally in the range from 0.4 to 3, preferably in the range from 0.6 to 1.8, particularly preferably in the range from 0.7 to 1.7.

In general, the temperature at the bottom of the first distillation apparatus is at least 20° C., preferably at least 50° C. and particularly preferably at least 70° C., and generally not more than 210° C., preferably not more than 190° C. The temperature in the first distillation apparatus is generally in the range from 20° C. to 210° C., preferably in the range from 50° C. to 190° C. The pressure in the first distillation apparatus is generally at least 0.001 MPa abs, preferably at least 0.005 MPa abs and particularly preferably at least 0.01 MPa abs, and generally not more than 1 MPa abs and preferably not more than 0.1 MPa abs. The pressure in the first distillation apparatus is generally in the range from 0.0001 MPa abs to 1 MPa abs, preferably in the range from 0.005 MPa abs to 0.1 MPa abs and particularly preferably in the range from 0.01 MPa abs to 0.1 MPa abs.

In the removal of the polar solvent in the first distillation apparatus, the formic acid-amine adduct (A2) and free tertiary amine (A1) can be obtained at the bottom of the first distillation apparatus, since formic acid-amine adducts having a low amine content are formed during the removal of the polar solvent. As a result, a bottoms mixture (S1) comprising the formic acid-amine adduct (A2) and the free tertiary amine (A1) is formed. The bottoms mixture (S1) comprises, depending on the amount of polar solvent separated off, the formic acid-amine adduct (A2) and possibly the free tertiary amine (A1) formed in the liquid phase of the first distillation apparatus. For further work-up, the bottoms mixture (S1) is optionally worked up further in process step (d) and subsequently fed to process step (e). It is also possible to feed the bottoms mixture (S1) from process step (c) directly to process step (e).

In the process step (d), the bottoms mixture (S1) obtained in step (c) can be separated into the upper phase (U2) and the lower phase (L2) in a second phase separation apparatus. The lower phase (L2) is subsequently worked up further according to process step (e). In a preferred embodiment, the upper phase (U2) from the second phase separation apparatus is recirculated to the hydrogenation reactor in step (a). In a further preferred embodiment, the upper phase (U2) from the second phase separation apparatus is recirculated to the extraction unit. What has been said in respect of the first phase separation apparatus, including preferences, applies analogously to process step (d) and the second phase separation apparatus.

In one embodiment, the process of the invention thus comprises the steps (a), (b1), (c), (d) and (e). In a further embodiment, the process of the invention comprises the steps (a), (b2), (c), (d) and (e). In a further embodiment, the process of the invention comprises the steps (a), (b3), (c), (d) and (e). In a further embodiment, the process of the invention comprises the steps (a), (b1), (c) and (e). In a further embodiment, the process of the invention comprises the steps (a), (b2), (c) and (e). In a further embodiment, the process of the invention comprises the steps (a), (b3), (c) and (e).

In one embodiment, the process of the invention consists of the steps (a), (b1), (c), (d) and (e). In a further embodiment, the process of the invention consists of the steps (a), (b2), (c), (d) and (e). In a further embodiment, the process of the invention consists of the steps (a), (b3), (c), (d) and (e). In a further embodiment, the process of the invention consists of the steps (a), (b1), (c) and (e). In a further embodiment, the process of the invention consists of the steps (a), (b2), (c) and (e). In a further embodiment, the process of the invention consists of the steps (a), (b3), (c) and (e).

### Dissociation of the Formic Acid-Amine Adduct (A2); Process Step (e)

The bottoms mixture (S1) obtained according to step (c) or the lower phase (L2) optionally obtained after the work-up according to step (d) is fed to a thermal dissociation unit for further reaction.

The formic acid-amine adduct (A2) comprised in the bottoms mixture (S1) or optionally in the lower phase (L2) is dissociated into formic acid and tertiary amine (A1) in the thermal dissociation unit. The carbon monoxide serving as decomposition inhibitor can here either be added to the feed or introduced directly in gaseous form into the thermal dissociation unit.

The formic acid is discharged from the thermal dissociation unit. The tertiary amine (A1) is recirculated to the hydrogenation reactor in step (a). The tertiary amine (A1) from the thermal dissociation unit can be recirculated directly to the hydrogenation reactor. It is also possible firstly to recirculate the tertiary amine (A1) from the thermal dissociation unit to the extraction unit in process step (b2) or process step (b3) and subsequently pass it on from the extraction unit to the hydrogenation reactor in step (a); this embodiment is preferred.

In a preferred embodiment, the thermal dissociation unit comprises a second distillation apparatus and a third phase separation apparatus, with the dissociation of the formic acid-amine adduct (A2) occurring in the second distillation apparatus to give a distillate (D2), which is discharged (taken off) from the second distillation apparatus, and a two-phase bottoms mixture (S2) comprising an upper phase (U3), which comprises the at least one tertiary amine (A1), and a lower phase (L3), which comprises the at least one formic acid-amine adduct (A2) and the at least one inhibitor.

The upper phase (U3) comprises, in a preferred embodiment, the inhibited complex catalyst and the free ligands in addition to the tertiary amine (A1).

The formic acid obtained in the second distillation apparatus can, for example, be taken off (i) via the top, (ii) via the top and via a side offtake or (iii) only via a side offtake from the second distillation apparatus. If the formic acid is taken off via the top, formic acid having a purity of up to 99.99% by weight is obtained. When it is taken off via a side offtake, aqueous formic acid is obtained, with a mixture comprising about 85% by weight of formic acid being particularly preferred here. Depending on the water content of the bottoms mixture (S1) or optionally the lower phase (L2) fed to the thermal dissociation unit, the formic acid can be taken off mostly as overhead product or mostly via the side offtake. If necessary, it is also possible to take off formic acid only via the side offtake, preferably with a formic acid content of about 85% by weight, in which case the required amount of water may also be provided by addition of additional water to the second distillation apparatus. The thermal dissociation of the formic acid-amine adduct (A2) is generally carried out at the process parameters in respect of pressure, temperature and configuration of the apparatus known from the prior art. These are described, for example, in EP 0 181 078 or WO 2006/021 411. Suitable second distillation apparatuses are, for example, distillation columns which generally comprise random packing elements, ordered packing and/or trays.

In general, the temperature at the bottom of the second distillation apparatus is at least 130° C., preferably at least 140° C. and particularly preferably at least 150° C., and generally not more than 210° C., preferably not more than 190° C., particularly preferably not more than 185° C. The pressure in the second distillation apparatus is generally at least 1 hPa abs, preferably at least 50 hPa abs and particularly preferably at least 100 hPa abs, and generally not more than 500 hPa, particularly preferably not more than 300 hPa abs and particularly preferably not more than 200 hPa abs.

The bottoms mixture (S2) obtained at the bottom of the second distillation apparatus is a two-phase mixture. In a preferred embodiment, the bottoms mixture (S2) is fed to the third phase separation apparatus of the thermal dissociation unit and separated there into the upper phase (U3), which comprises the tertiary amine (A1), the inhibited complex catalyst and the free ligands, and the lower phase (L3), which comprises the formic acid-amine adduct (A2) and the inhibitor. The upper phase (U3) is discharged from the third phase separation apparatus of the thermal dissociation unit and recirculated to the hydrogenation reactor in step (a). The recirculation can be carried out directly to the hydrogenation reactor in step (a) or the upper phase (U3) is firstly fed to the extraction unit in step (b2) or step (b3) and from there passed on to the hydrogenation reactor in step (a). The lower phase (L3) obtained in the third phase separation apparatus is then fed back into the second distillation apparatus of the thermal dissociation unit. The formic acid-amine adduct (A2) comprised in the lower phase (L3) is again subjected to dissociation in the second distillation apparatus, once again with formic acid and free tertiary amine (A1) being obtained and with a two-phase bottoms mixture (S2) again being formed at the bottom of the second distillation apparatus of the thermal dissociation unit and then being fed again to the third phase separation apparatus of the thermal dissociation unit for further work-up.

The carbon monoxide-inhibited catalyst is present in enriched form in the upper phase (U3). The inhibited complex catalyst comprised in the upper phase (U3) is, in one embodiment, reconverted into the active form under the conditions of the hydrogenation in step (a) after recirculation to the hydrogenation reactor.

In a further embodiment, the inhibited catalyst is subjected to a thermal treatment at temperatures in the range from 100 to 200° C. in the absence of a carbon monoxide partial pressure before recirculation to step (a). For the purposes of the present invention, “absence of a carbon monoxide partial pressure” means that in the reactivation of the inhibited catalyst only the carbon monoxide which is bound to the inhibited complex catalyst or is eliminated from the inhibited complex catalyst and replaced by free ligands in the reactivation is present.

The introduction of the bottoms mixture (S1) or optionally of the lower phase (L2) into the thermal dissociation unit in process step (e) can be effected into the second distillation apparatus and/or the third phase separation apparatus. In a preferred embodiment, the bottoms mixture (S1) or optionally the lower phase (L2) is introduced into the second distillation apparatus of the thermal separation unit. In a further embodiment, the bottoms mixture (S1) or optionally the lower phase (L2) is introduced into the third phase separation vessel of the thermal dissociation unit.

In a further embodiment, the bottoms mixture (S1) or optionally the lower phase (L2) is introduced both into the second distillation apparatus of the thermal dissociation unit and into the third phase separation apparatus of the thermal dissociation unit. For this purpose, the bottoms mixture (S1) or optionally the lower phase (L2) is divided into two substreams of which one substream is introduced into the second distillation apparatus and one substream is introduced into the third separation apparatus of the thermal dissociation unit.

The invention is illustrated by the following drawings and examples without being limited thereto.

The drawings show in detail:

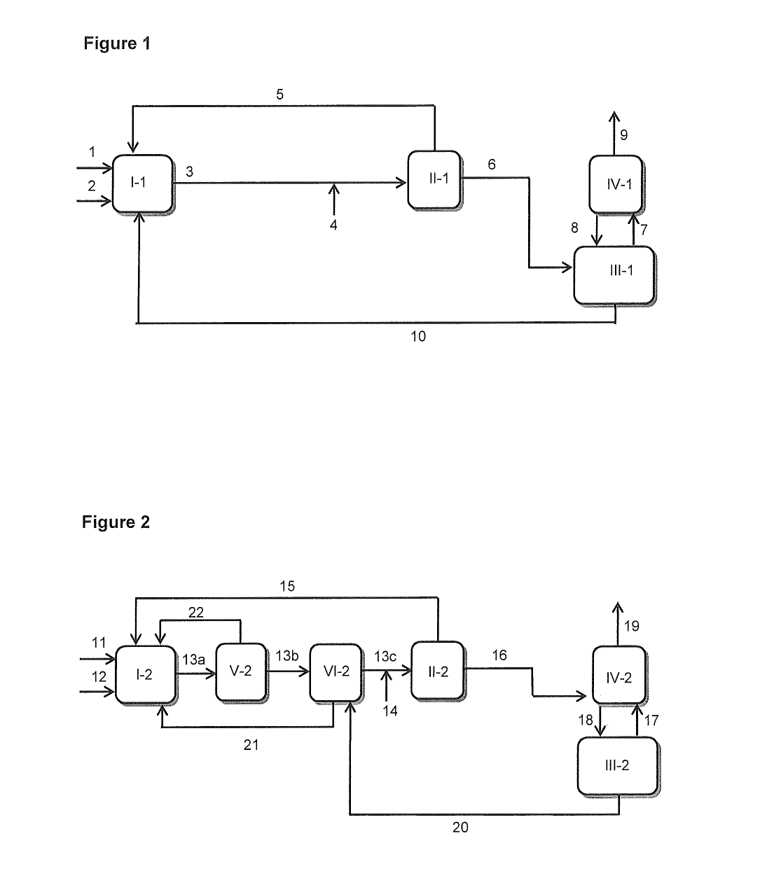

FIG. 1 a block diagram of a preferred embodiment of the process of the invention,

FIG. 2 a block diagram of a further preferred embodiment of the process of the invention,

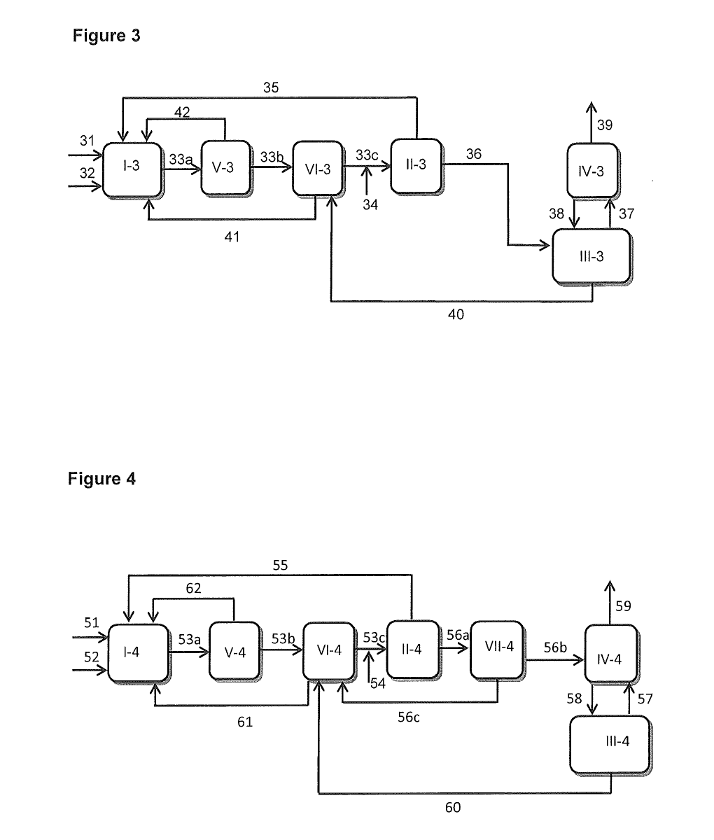

FIG. 3 a block diagram of a further preferred embodiment of the process of the invention,

FIG. 4 a block diagram of a further preferred embodiment of the process of the invention,

FIG. 5 a block diagram of a further preferred embodiment of the process of the invention,

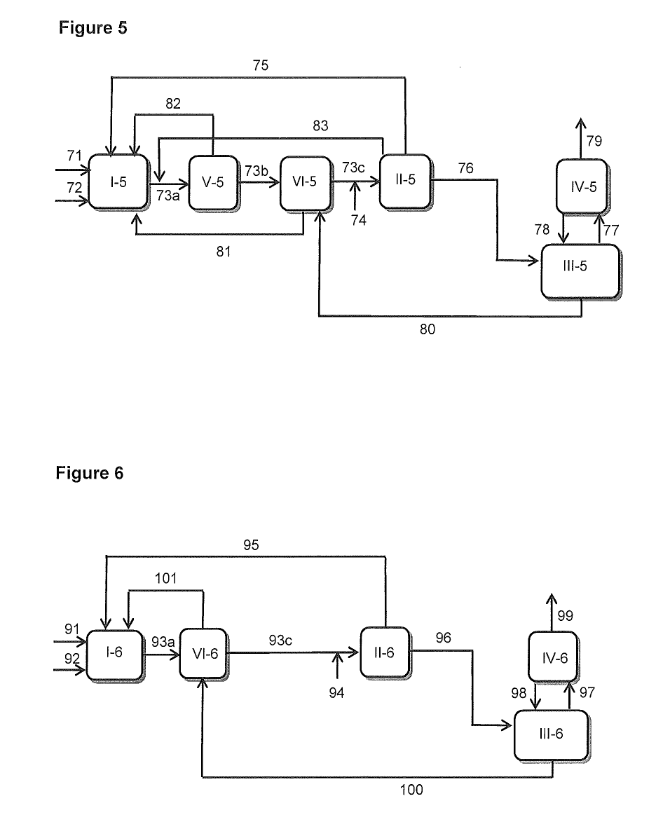

FIG. 6 a block diagram of a further preferred embodiment of the process of the invention.

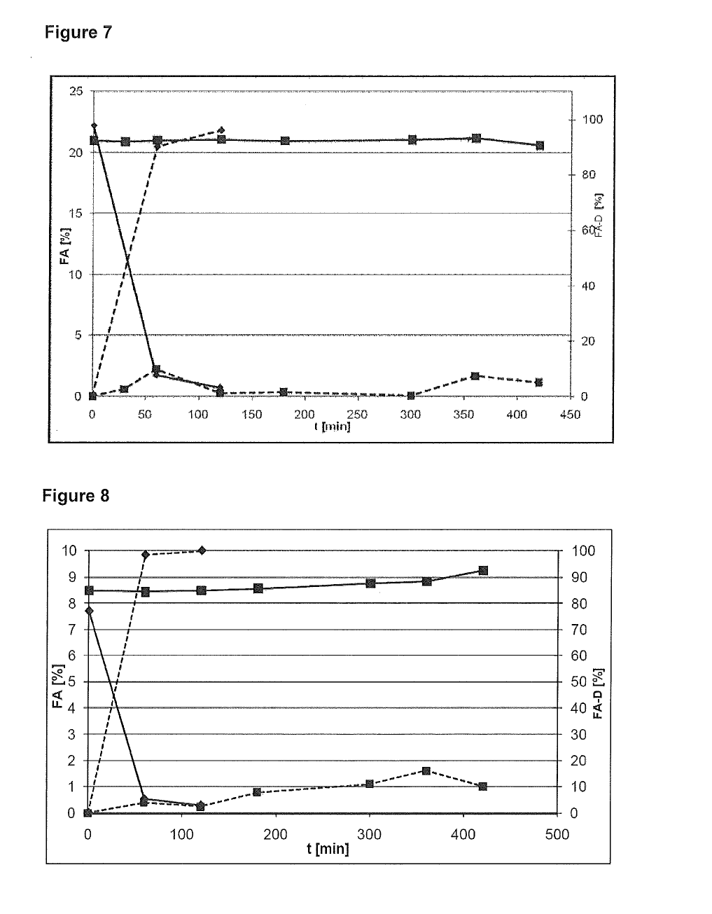

FIGS. 7, 8, 9 and 10 graphical representation of the inhibition experiments H1, H2, H3 and H4.

In FIGS. 1 to 6, the reference numerals have the following meanings:

### FIG. 1

I-1 hydrogenation reactor
II-1 first distillation apparatus
III-1 third phase separation apparatus (of the thermal dissociation unit)
IV-1 second distillation apparatus (of the thermal dissociation unit)
1 stream comprising carbon dioxide
2 stream comprising hydrogen
3 stream comprising formic acid-amine adduct ((A2), residues of the catalyst, polar solvent; (lower phase (L1))
4 carbon monoxide stream
5 stream comprising polar solvent; (distillate (D1))
6 stream comprising tertiary amine (A1) (upper phase (U2)) and formic acid-amine adduct (A2) (lower phase (L2)); bottoms mixture (S1)
7 stream comprising formic acid-amine adduct (A2) and inhibitor; lower phase (L3)
8 stream comprising tertiary amine (A1) (upper phase (U3)) and also formic acid-amine adduct (A2) and inhibitor (lower phase (L3)); bottoms mixture (S2)
9 stream comprising formic acid; (distillate (D2))
10 stream comprising tertiary amine (A1); upper phase (U3)

### FIG. 2

I-2 hydrogenation reactor
II-2 first distillation apparatus
III-2 third phase separation apparatus (of the thermal dissociation unit)
IV-2 second distillation apparatus (of the thermal dissociation unit)
V-2 first phase separation apparatus
VI-2 extraction unit
11 stream comprising carbon dioxide
12 stream comprising hydrogen
13a stream comprising hydrogenation mixture (H)
13b stream comprising lower phase (L1)
13c stream comprising raffinate (R2)
14 stream comprising carbon monoxide
15 stream comprising distillate (D1)
16 stream comprising bottoms mixture (S1)
17 stream comprising lower phase (L3)
18 stream comprising bottoms mixture (S2)
19 stream comprising formic acid; (distillate (D2))
20 stream comprising upper phase (U3)
21 stream comprising extract (E2)
22 stream comprising upper phase (U1)

### FIG. 3

I-3 hydrogenation reactor
II-3 first distillation apparatus
III-3 third phase separation apparatus (of the thermal dissociation unit)
IV-3 second distillation apparatus (of the thermal dissociation unit)
V-3 first phase separation apparatus
VI-3 extraction unit
31 stream comprising carbon dioxide
32 stream comprising hydrogen
33a stream comprising hydrogenation mixture (H)
33b stream comprising lower phase (L1)
33c stream comprising raffinate (R2)
34 stream comprising carbon monoxide
35 stream comprising distillate (D1)
36 stream comprising bottoms mixture (S1)
37 stream comprising lower phase (L3)
38 stream comprising bottoms mixture (S2)
39 stream comprising formic acid; (distillate (D2))
40 stream comprising upper phase (U3)
41 stream comprising extract (E2)
42 stream comprising upper phase (U1)

### FIG. 4

I-4 hydrogenation reactor
II-4 first distillation apparatus
III-4 third phase separation apparatus (of the thermal dissociation unit)
IV-4 second distillation apparatus (of the thermal dissociation unit)
V-4 first phase separation apparatus
VI-4 extraction unit
VII-4 second phase separation apparatus
51 stream comprising carbon dioxide
52 stream comprising hydrogen
53a stream comprising hydrogenation mixture (H)
53b stream comprising lower phase (L1)
53c stream comprising raffinate (R2)
54 stream comprising carbon monoxide
55 stream comprising distillate (D1)
56a stream comprising bottoms mixture (S1)
56b stream comprising lower phase (L2)
56c stream comprising upper phase (U2)
57 stream comprising lower phase (L3)
58 stream comprising bottoms mixture (S2)
59 stream comprising formic acid; (distillate (D2))
60 stream comprising upper phase (U3)
61 stream comprising extract (E2)
62 stream comprising upper phase (U1)

### FIG. 5

I-5 hydrogenation reactor
II-5 first distillation apparatus
III-5 third phase separation apparatus (of the thermal dissociation unit)
IV-5 second distillation apparatus (of the thermal dissociation unit)
V-5 first phase separation apparatus
VI-5 extraction unit
71 stream comprising carbon dioxide
72 stream comprising hydrogen
73a stream comprising hydrogenation mixture (H)
73b stream comprising lower phase (L1)
73c stream comprising raffinate (R2)
74 stream comprising carbon monoxide
75 stream comprising low-water distillate (D1wa)
76 stream comprising bottoms mixture (S1)
77 stream comprising lower phase (L3)
78 stream comprising bottoms mixture (S2)
79 stream comprising formic acid; (distillate (D2))
80 stream comprising upper phase (U3)
81 stream comprising extract (E2)
82 stream comprising upper phase (U1)
83 stream comprising water-rich distillate (D1wr)

### FIG. 6

I-6 hydrogenation reactor
II-6 first distillation apparatus
III-6 third phase separation apparatus (of the thermal dissociation unit)
IV-6 second distillation apparatus (of the thermal dissociation unit)
VI-6 extraction unit
91 stream comprising carbon dioxide
92 stream comprising hydrogen
93a stream comprising hydrogenation mixture (H)
93c stream comprising raffinate (R1)
94 stream comprising carbon monoxide
95 stream comprising distillate (D1)
96 stream comprising bottoms mixture (S1)
97 stream comprising lower phase (L3)
98 stream comprising bottoms mixture (S2)
99 stream comprising formic acid; (distillate (D2))
100 stream comprising upper phase (U3)
101 stream comprising extract (E1)

### FIGS. 7, 8, 9 and 10

t[min] time of experiment in minutes
FA[%] % by weight of formic acid in the form of the formic acid-amine adduct (A3) based on the total weight of the formic acid used in the form of the formic acid-amine adduct (A3)
FA-D[%] % by weight of the decomposed formic acid based on the total weight of the formic acid used in the form of the formic acid-amine adduct (A3)
[symbol] values for FA[%] with inhibitor (passage of carbon monoxide)
[symbol] values for FA-D[%] with inhibitor (passage of carbon monoxide)
[symbol] values for FA[%] without inhibitor
[symbol] values for FA-D[%] without inhibitor

In the embodiment of FIG. 1, a stream 1 comprising carbon dioxide and a stream 2 comprising hydrogen are fed to a hydrogenation reactor I-1. It is possible to feed further streams (not shown) to the hydrogenation reactor I-1 in order to compensate any losses of the tertiary amine (A1) or the complex catalyst.

In the hydrogenation reactor I-1, carbon dioxide and hydrogen are reacted in the presence of a tertiary amine (A1), a polar solvent and a complex catalyst comprising at least one element of groups 8, 9 and 10 of the Periodic Table. This gives a two-phase hydrogenation mixture (H) which comprises an upper phase (U1) comprising the complex catalyst and the tertiary amine (A1) and a lower phase (L1) comprising the polar solvent, residues of the complex catalyst and the formic acid-amine adduct (A2).

The lower phase (L1) is fed as stream 3 to the distillation apparatus II-1. The upper phase (U1) remains in the hydrogenation reactor I-1. In the embodiment of FIG. 1, the hydrogenation reactor I-1 simultaneously serves as first phase separation apparatus.

The inhibitor is added continuously or discontinuously as stream 4 to the stream 3. In the first distillation apparatus II-1, the lower phase (L1) is separated into a distillate (D1) comprising the polar solvent, which is recirculated as stream 5 to the hydrogenation reactor I-1, and a two-phase bottoms mixture (S1) comprising an upper phase (U2), which comprises the tertiary amine (A1) and the inhibited complex catalyst, and the lower phase (L2), which comprises the formic acid-amine adduct (A2).

The bottoms mixture (S1) is fed as stream 6 to the third phase separation apparatus III-1 of the thermal dissociation unit.

In the third phase separation apparatus III-1 of the thermal dissociation unit, the bottoms mixture (S1) is separated to give an upper phase (U3), which comprises the tertiary amine (A1) and the inhibited complex catalyst, and a lower phase (L3), which comprises the formic acid-amine adduct (A2).

The upper phase (U3) is recirculated as stream 10 to the hydrogenation reactor I-1. The lower phase (L3) is fed as stream 7 to the second distillation apparatus IV-1 of the thermal dissociation unit. The formic acid-amine adduct (A2) comprised in the lower phase (L3) is dissociated into formic acid and free tertiary amine (A1) in the second distillation apparatus IV-1. A distillate (D2) and a two-phase bottoms mixture (S2) are obtained in the second distillation apparatus IV-1.

The distillate (D2) comprising formic acid is discharged as stream 9 from the distillation apparatus IV-1. The two-phase bottoms mixture (S2) comprising the upper phase (U3), which comprises the tertiary amine (A1), and the lower phase (L3), which comprises the formic acid-amine adduct (A2), is recirculated as stream 8 to the third phase separation apparatus III-1 of the thermal dissociation unit. In the third phase separation apparatus III-1, the bottoms mixture (S2) is separated into upper phase (U3) and lower phase (L3). The upper phase (U3) is recirculated as stream 10 to the hydrogenation reactor I-1. The lower phase (L3) is recirculated as stream 7 to the second distillation apparatus IV-1.

In the embodiment of FIG. 2, a stream 11 comprising carbon dioxide and a stream 12 comprising hydrogen are fed to a hydrogenation reactor I-2. It is possible to feed further streams (not shown) to the hydrogenation reactor I-2 in order to compensate any losses of the tertiary amine (A1) or the complex catalyst.

In the hydrogenation reactor I-2, carbon dioxide and hydrogen are reacted in the presence of a tertiary amine (A1), a polar solvent and a complex catalyst comprising at least one element of groups 8, 9 and 10 of the Periodic Table. This gives a two-phase hydrogenation mixture (H) which comprises an upper phase (U1) comprising the complex catalyst and the tertiary amine (A1) and a lower phase (L1) comprising the polar solvent, residues of the complex catalyst and the formic acid-amine adduct (A2).

The hydrogenation mixture (H) is fed as stream 13a to a first phase separation apparatus V-2. In the first separation phase apparatus V-2, the hydrogenation mixture (H) is separated into the upper phase (U1) and the lower phase (L1).

The upper phase (U1) is recirculated as stream 22 to the hydrogenation reactor I-2. The lower phase (L1) is fed as stream 13b to the extraction unit VI-2. In this, the lower phase (L1) is extracted with the tertiary amine (A1) which is recirculated as stream 20 (upper phase (U3)) from the third phase separation apparatus III-2 to the extraction apparatus VI-2.

A raffinate (R2) and an extract (E2) are obtained in the extraction unit VI-2. The raffinate (R2) comprises the formic acid-amine adduct (A2) and the polar solvent and is fed as stream 13c to the first distillation apparatus II-2. The extract (E2) comprises the tertiary amine (A1) and the residues of the complex catalyst and is recirculated as stream 21 to the hydrogenation reactor I-2.

The inhibitor is added continuously or discontinuously as stream 14 to the stream 13c. In the first distillation apparatus II-2, the raffinate (R2) is separated into a distillate (D1) comprising the polar solvent, which is recirculated as stream 15 to the hydrogenation reactor I-2, and a two-phase bottoms mixture (S1).

The bottoms mixture (S1) comprises an upper phase (U2), which comprises the tertiary amine (A1) and the inhibited complex catalyst, and a lower phase (L2), which comprises the formic acid-amine adduct (A2). The bottoms mixture (S1) is fed as stream 16 to the second distillation apparatus IV-2.

The formic acid-amine adduct comprised in the bottoms mixture (S1) is dissociated into formic acid and free tertiary amine (A1) in the second distillation apparatus IV-2. A distillate (D2) and a bottoms mixture (S2) are obtained in the second distillation apparatus IV-2.

The distillate (D2) comprising formic acid is discharged as stream 19 from the second distillation apparatus IV-2. The two-phase bottoms mixture (S2) comprising the upper phase (U3), which comprises the tertiary amine (A1) and the inhibited complex catalyst, and the lower phase (L3), which comprises the formic acid-amine adduct (A2), is recirculated as stream 18 to the third phase separation apparatus III-2 of the thermal dissociation unit.

In the third phase separation apparatus III-2 of the thermal dissociation unit, the bottoms mixture (S2) is separated to give an upper phase (U3) comprising the tertiary amine (A1) and the inhibited complex catalyst and a lower phase (L3) comprising the formic acid-amine adduct (A2).

The upper phase (U3) from the third phase separation apparatus III-2 is recirculated as stream 20 to the extraction unit VI-2. The lower phase (L3) is fed as stream 17 to the second distillation apparatus IV-2 of the thermal dissociation unit. The formic acid-amine adduct (A2) comprised in the lower phase (L3) is dissociated into formic acid and free tertiary amine (A1) in the second distillation apparatus IV-2. As indicated above, a distillate (D2) and a bottoms mixture (S2) are then again obtained in the second distillation apparatus IV-2.

In the embodiment of FIG. 3, a stream 31 comprising carbon dioxide and a stream 32 comprising hydrogen are fed to a hydrogenation reactor I-3. It is possible to feed further streams (not shown) to the hydrogenation reactor I-3 in order to compensate any losses of the tertiary amine (A1) or the complex catalyst.

In the hydrogenation reactor I-3, carbon dioxide and hydrogen are reacted in the presence of a tertiary amine (A1), a polar solvent and a complex catalyst comprising at least one element of groups 8, 9 and 10 of the Periodic Table. This gives a two-phase hydrogenation mixture (H) which comprises an upper phase (U1) comprising the complex catalyst and the tertiary amine (A1) and a lower phase (L1) comprising the polar solvent, residues of the complex catalyst and the formic acid-amine adduct (A2).

The hydrogenation mixture (H) is fed as stream 33a to a first phase separation apparatus V-3. In the first phase separation apparatus V-3, the hydrogenation mixture (H) is separated into the upper phase (U1) and the lower phase (L1).

The upper phase (U1) is recirculated as stream 42 to the hydrogenation reactor I-3. The lower phase (L1) is fed as stream 33b to the extraction unit VI-3. In this, the lower phase (L1) is extracted with the tertiary amine (A1) which is recirculated as stream 40 (upper phase (U3)) from the third phase separation apparatus III-3 of the thermal dissociation unit to the extraction unit VI-3.

A raffinate (R2) and an extract (E2) are obtained in the extraction unit VI-3. The raffinate (R2) comprises the formic acid-amine adduct (A2) and the polar solvent and is fed as stream 33c to the first distillation apparatus II-3. The extract (E2) comprises the tertiary amine (A1) and the residues of the complex catalyst and is recirculated as stream 41 to the hydrogenation reactor I-2.

The inhibitor is added continuously or discontinuously as stream 34 to the stream 33c. In the first distillation apparatus II-3, the raffinate (R2) is separated into a distillate (D1) comprising the polar solvent, which is recirculated as stream 35 to the hydrogenation reactor I-3, and a two-phase bottoms mixture (S1).

The bottoms mixture (S1) comprises an upper phase (U2), which comprises the tertiary amine (A1) and the inhibited complex catalyst, and a lower phase (L2), which comprises the formic acid-amine adduct (A2).

The bottoms mixture (S1) is fed as stream 36 to the third phase separation apparatus III-3 of the thermal dissociation unit.

In the third phase separation apparatus III-3 of the thermal dissociation unit, the bottoms mixture (S1) is separated to give an upper phase (U3) comprising the tertiary amine (A1) and the inhibited complex catalyst and a lower phase (L3) comprising the formic acid-amine adduct (A2).

The upper phase (U3) is recirculated as stream 40 to the extraction unit VI-3. The lower phase (L3) is fed as stream 37 to the second distillation apparatus IV-3 of the thermal dissociation unit. The formic acid-amine adduct (A2) comprised in the lower phase (L3) is dissociated into formic acid and free tertiary amine (A1) in the second distillation apparatus IV-3. A distillate (D2) and a bottoms mixture (S2) are obtained in the second distillation apparatus IV-3.

The distillate (D2) comprising formic acid is discharged as stream 39 from the distillation apparatus IV-3. The two-phase bottoms mixture (S2) comprising the upper phase (U3), which comprises the tertiary amine (A1), and the lower phase (L3), which comprises the formic acid-amine adduct (A2), is recirculated as stream 38 to the third phase separation apparatus III-3 of the thermal dissociation unit. In the third phase separation apparatus III-3, the bottoms mixture (S2) is separated. The upper phase (U3) is recirculated to the extraction unit VI-3. The lower phase (L3) is recirculated to the second distillation apparatus IV-3.

In the embodiment of FIG. 4, a stream 51 comprising carbon dioxide and a stream 52 comprising hydrogen are fed to a hydrogenation reactor I-4. It is possible to feed further streams (not shown) to the hydrogenation reactor I-4 in order to compensate any losses of the tertiary amine (A1) or the complex catalyst.

In the hydrogenation reactor I-4, carbon dioxide and hydrogen are reacted in the presence of a tertiary amine (A1), a polar solvent and a complex catalyst comprising at least one element of groups 8, 9 and 10 of the Periodic Table. This gives a two-phase hydrogenation mixture (H) which comprises an upper phase (U1) comprising the complex catalyst and the tertiary amine (A1) and a lower phase (L1) comprising the polar solvent, residues of the complex catalyst and the formic acid-amine adduct (A2).

The hydrogenation mixture (H) is fed as stream 53a to a first phase separation apparatus V-4. In the first phase separation apparatus V-4, the hydrogenation mixture (H) is separated into the upper phase (U1) and the lower phase (L1).

The upper phase (U1) is recirculated as stream 62 to the hydrogenation reactor I-4. The lower phase (L1) is fed as stream 53b to the extraction unit VI-4. In this, the lower phase (L1) is extracted with the tertiary amine (A1) which is recirculated as stream 60 (upper phase (U3)) from the third phase separation apparatus III-4 of the thermal dissociation unit and as stream 56c from the second phase separation apparatus VII-4 to the extraction unit VI-4.

A raffinate (R2) and an extract (E2) are obtained in the extraction unit VI-4. The raffinate (R2) comprises the formic acid-amine adduct (A2) and the polar solvent and is fed as stream 53c to the first distillation apparatus II-4. The extract (E2) comprises the tertiary amine (A1) and the residues of the complex catalyst and is recirculated as stream 61 to the hydrogenation reactor I-4.

The inhibitor is added continuously or discontinuously as stream 54 to the stream 53c. In the first distillation apparatus II-4, the raffinate (R2) is separated into a distillate (D1) comprising the polar solvent, which is recirculated as stream 55 to the hydrogenation reactor I-4, and a two-phase bottoms mixture (S1).

The bottoms mixture (S1) comprises an upper phase (U2), which comprises the tertiary amine (A1) and the inhibited complex catalyst, and a lower phase (L2), which comprises the formic acid-amine adduct (A2). The bottoms mixture (S1) is fed as stream 56a to the second phase separation apparatus VII-4.

In the second phase separation apparatus VII-4, the bottoms mixture (S1) is separated into the upper phase (U2) and the lower phase (L2). The upper phase (U2) is recirculated from the second phase separation apparatus VII-4 as stream 56c to the extraction unit VI-4.

The lower phase (L2) is fed as stream 56b to the second distillation apparatus IV-4.

The formic acid-amine adduct (A2) comprised in the lower phase (L2) is dissociated into formic acid and free tertiary amine (A1) in the second distillation apparatus IV-4. A distillate (D2) and a bottoms mixture (S2) are obtained in the second distillation apparatus IV-4.

The distillate (D2) comprising formic acid is discharged as stream 59 from the second distillation apparatus IV-4. The two-phase bottoms mixture (S2) comprising the upper phase (U3), which comprises the tertiary amine (A1) and the inhibited complex catalyst, and the lower phase (L3), which comprises the formic acid-amine adduct (A2), is recirculated as stream 58 to the third phase separation apparatus III-4 of the thermal dissociation unit.

In the third phase separation apparatus III-4 of the thermal dissociation unit, the bottoms mixture (S2) is separated to give an upper phase (U3) comprising the tertiary amine (A1) and the inhibited complex catalyst and a lower phase (L3) comprising the formic acid-amine adduct (A2).

The upper phase (U3) is recirculated from the third phase separation apparatus III-4 as stream 60 to the extraction unit VI-4. The lower phase (L3) is fed as stream 57 to the second distillation apparatus IV-4 of the thermal dissociation unit. The formic acid-amine adduct (A2) comprised in the lower phase (L3) is dissociated into formic acid and free tertiary amine (A1) in the second distillation apparatus IV-4. A distillate (D2) and a bottoms mixture (S2) are, as indicated above, then again obtained in the second distillation apparatus IV-4.

In the embodiment of FIG. 5, a stream 71 comprising carbon dioxide and a stream 72 comprising hydrogen are fed to a hydrogenation reactor I-5. It is possible to feed further streams (not shown) to the hydrogenation reactor I-5 in order to compensate any losses of the tertiary amine (A1) or the complex catalyst.

In the hydrogenation reactor I-5, carbon dioxide and hydrogen are reacted in the presence of a tertiary amine (A1), a polar solvent and a complex catalyst comprising at least one element of groups 8, 9 and 10 of the Periodic Table. This gives a two-phase hydrogenation mixture (H) which comprises an upper phase (U1) comprising the complex catalyst and the tertiary amine (A1) and a lower phase (L1) comprising the polar solvent, residues of the complex catalyst and the formic acid-amine adduct (A2).

The hydrogenation mixture (H) is fed as stream 73a to a first phase separation apparatus V-5. In the first phase separation apparatus V-5, the hydrogenation mixture (H) is separated into the upper phase (U1) and the lower phase (L1).

The upper phase (U1) is recirculated as stream 82 to the hydrogenation reactor I-5. The lower phase (L1) is fed as stream 73b to the extraction unit VI-5. Here, the lower phase (L1) is extracted with the tertiary amine (A1) which is recirculated as stream 80 (upper phase (U3)) from the third phase separation apparatus of the thermal dissociation unit to the extraction unit VI-5.

A raffinate (R2) and an extract (E2) are obtained in the extraction unit VI-5. The raffinate (R2) comprises the formic acid-amine adduct (A2) and the polar solvent and is fed as stream 73c to the first distillation apparatus II-5. The extract (E2) comprises the tertiary amine (A1) and the residues of the complex catalyst and is recirculated as stream 81 to the hydrogenation reactor I-5.

The inhibitor is added continuously or discontinuously as stream 74 to the stream 73c. In the first distillation apparatus II-5, the raffinate (R2) is separated into a water-rich distillate (D1wr), a low-water distillate (D1wa) and a two-phase bottoms mixture (S1). The water-rich distillate (D1wr) is added as stream 83 to the stream 73a. The low-water distillate (D1wa) is recirculated as stream 75 to the hydrogenation reactor I-5. A prerequisite of the embodiment of FIG. 5 is that a mixture of one or more alcohols with water is used as polar solvent.

The bottoms mixture (S1) comprises an upper phase (U2), which comprises the tertiary amine (A1) and the inhibited complex catalyst, and a lower phase (L2), which comprises the formic acid-amine adduct (A2).

The bottoms mixture (S1) is fed as stream 76 to the third phase separation apparatus III-5 of the thermal dissociation unit.

In the third phase separation apparatus III-5 of the thermal dissociation unit, the bottoms mixture (S1) is separated to give an upper phase (U3) comprising the tertiary amine (A1) and the inhibited complex catalyst and a lower phase (L3) comprising the formic acid-amine adduct (A2).

The upper phase (U3) is recirculated as stream 80 to the extraction unit IV-5. The lower phase (L3) is fed as stream 77 to the second distillation apparatus IV-5 of the thermal dissociation unit. The formic acid-amine adduct (A2) comprised in the lower phase (L3) is dissociated into formic acid and free tertiary amine (A1) in the second distillation apparatus IV-5. A distillate (D2) and a bottoms mixture (S2) are obtained in the second distillation apparatus IV-5.

The distillate (D2) comprising formic acid is discharged as stream 79 from the distillation apparatus IV-5. The two-phase bottoms mixture (S2) comprising the upper phase (U3), which comprises the tertiary amine (A1) and the inhibited complex catalyst, and the lower phase (L3), which comprises the formic acid-amine adduct (A2), is recirculated as stream 78 to the third phase separation apparatus III-5 of the thermal dissociation unit. The bottoms mixture (S2) is separated in the third phase separation apparatus III-5. The upper phase (U3) is recirculated as stream 80 to the extraction unit VI-5. The lower phase (L3) is recirculated as stream 77 to the second distillation apparatus IV-5.

In the embodiment of FIG. 6, a stream 91 comprising carbon dioxide and a stream 92 comprising hydrogen are fed to a hydrogenation reactor I-6. It is possible to feed further streams (not shown) to the hydrogenation reactor I-6 in order to compensate any losses of the tertiary amine (A1) or the complex catalyst.

In the hydrogenation reactor I-6, carbon dioxide and hydrogen are reacted in the presence of a tertiary amine (A1), a polar solvent and a complex catalyst comprising at least one element of groups 8, 9 and 10 of the Periodic Table. This gives a two-phase hydrogenation mixture (H) which comprises an upper phase (U1) comprising the complex catalyst and the tertiary amine (A1) and a lower phase (L1) comprising the polar solvent, residues of the complex catalyst and the formic acid-amine adduct (A2).

The hydrogenation mixture (H) is fed as stream 93a to the extraction unit VI-6.

In this, the hydrogenation mixture (H) is extracted with the tertiary amine (A1) which is recirculated as stream 100 (upper phase (U3)) from the third phase separation apparatus III-6 of the thermal dissociation unit to the extraction unit VI-6.

A raffinate (R1) and an extract (E1) are obtained in the extraction unit VI-6. The raffinate (R1) comprises the formic acid-amine adduct (A2) and the polar solvent and is fed as stream 93c to the first distillation apparatus II-6. The extract (E1) comprises the tertiary amine (A1) and the complex catalyst and is recirculated as stream 101 to the hydrogenation reactor I-6.

The inhibitor is added continuously or discontinuously as stream 94 to the stream 93c. In the first distillation apparatus II-6, the raffinate (R1) is separated into a distillate (D1) comprising the polar solvent, which is recirculated as stream 95 to the hydrogenation reactor I-6, and a two-phase bottoms mixture (S1).

The bottoms mixture (S1) comprises an upper phase (U2), which comprises the tertiary amine (A1) and the inhibited complex catalyst, and a lower phase (L2), which comprises the formic acid-amine adduct (A2).

The bottoms mixture (S1) is fed as stream 96 to the third phase separation apparatus III-6 of the thermal dissociation unit.

In the third phase separation apparatus III-6 of the thermal dissociation unit, the bottoms mixture (S1) is separated to give an upper phase (U3) comprising the tertiary amine (A1) and the inhibited complex catalyst and a lower phase (L3) comprising the formic acid-amine adduct (A2).

The upper phase (U3) is recirculated as stream 100 to the extraction unit VI-6. The lower phase (L3) is fed as stream 97 to the second distillation apparatus IV-6 of the thermal dissociation unit. The formic acid-amine adduct (A2) comprised in the lower phase (L3) is dissociated into formic acid and free tertiary amine (A1) in the second distillation apparatus IV-6. A distillate (D2) and a bottoms mixture (S2) are obtained in the second distillation apparatus IV-6.

The distillate (D2) comprising formic acid is discharged as stream 99 from the distillation apparatus IV-6. The two-phase bottoms mixture (S2) comprising the upper phase (U3), which comprises the tertiary amine (A1) and the inhibited complex catalyst, and the lower phase (L3), which comprises the formic acid-amine adduct (A2), is recirculated as stream 98 to the third phase separation apparatus III-6 of the thermal dissociation unit. The bottoms mixture (S2) is separated in the third phase separation apparatus III-6. The upper phase (U3) is recirculated as stream 100 to the extraction unit VI-6. The lower phase (L3) is recirculated as stream 97 to the second distillation apparatus IV-6.

The invention is illustrated below by means of examples and a drawing.

### EXAMPLES

### Examples A-1 to A-6

### According to the Invention (Hydrogenation and Phase Separation, Work-Up of the Output from the Hydrogenation Reactor)

A 250 ml Hastelloy C autoclave equipped with a magnetic stirrer bar was charged under inert conditions with tertiary amine (A1), polar solvent and complex catalyst. The autoclave was subsequently closed and CO₂ was injected at room temperature. H₂ was then injected and the reactor was heated while stirring (700 rpm). After the desired reaction time, the autoclave was cooled and the hydrogenation mixture (H) was depressurized. A two-phase hydrogenation mixture (H) was obtained, with the upper phase (U1) being enriched in the still free tertiary amine (A1) and the complex catalyst and the lower phase (L1) being enriched in the polar solvent and the formic acid-amine adduct (A2) formed. The total content of formic acid in the formic acid-amine adduct (A2) was determined by potentiometric titration with 0.1 N KOH in MeOH using a “Mettler Toledo DL50” titrator. The turnover frequency (=TOF; for the definition of the TOF see: J. F. Hartwig, Organotransition Metal Chemistry, 1^{st} edition, 2010, University Science Books, Sausalito/Calif. p. 545) and the reaction rate were calculated therefrom. The composition of the two phases was determined by gas chromatography. The ruthenium content was determined by atomic adsorption spectroscopy (=AAS). The parameters and results of the individual experiments are shown in Table 1.1.

Examples A-1 to A-6 show that high to very high reaction rates of up to 0.98 mol kg⁻¹ h⁻¹ are achieved in the process of the invention even with variation of the tertiary amine (A1), the polar solvent, the complex catalyst in respect of the ligands and the metal component, the amount of the catalyst and the amount of water added. All systems examined formed two phases, with the upper phase (U1) in each case being enriched in the still free tertiary amine (A1) and the complex catalyst and the lower phase (L1) in each case being enriched in the polar solvent and the formic acid-amine adduct (A2) formed.

k_{Ru}, (c_{Ru} in upper phase (U1)/c_{Ru} in lower phase (L1)) is the partition coefficient of the metal component of the complex catalyst between the upper phase (U1) and the lower phase (L1). c_{Ru} in lower phase (U1) is the concentration of the metal component of the complex catalyst in the upper phase (U1), while c_{Ru} in lower phase (L1) is the concentration of the metal component of the complex catalyst in the lower phase (L1).

TABLE 1.1

| | Example A-1 | Example A-2 | Example A-3 | Example A-4 | Example A-5 | Example A-6 |
|---|---|---|---|---|---|---|
| Tertiary | 75 g of trihexylamine | 75 g of tripentylamine | 75 g of tripentylamine | 75 g of tripentylamine | 75 g of tripentylamine | 75 g of trihexylamine |
| amine (A1) | | | | | | |
| Polar | 17.8 g of 1-propanol | 17.8 g of 1-propanol | 18.8 g of 1-propanol | 24.0 g of 1-propanol | 22.0 g of methanol | 25.0 g of ethanol |
| solvent | 7.3 g of water | 7.3 g of water | 6.3 g of water | 1.0 g of water | 3.0 g of water | 8.0 g of water |
| (used) | | | | | | |
| Complex | 0.2 g of | 0.2 g of | 0.2 g of | 0.16 g of | 0.16 g of | 0.16 g of |
| catalyst | [Ru(PⁿBu₃)₄(H)₂] | [Ru(PⁿBu₃)₄(H)₂] | [Ru(PⁿBu₃)₄(H)₂] | [Ru(PⁿOct₃)₄(H)₂] | [Ru(PⁿOct₃)₄(H)₂] | [Ru(PⁿBu₃)₄(H)₂] |
| | | | | | 0.08 g 1,2- | 0.08 g 1,2- |
| | | | | | bis(dicyclohexyl- | bis(dicyclohexyl- |
| | | | | | phosphino)ethane | phophino)ethane |
| Injection | 19.6 g of 2.4 | 20.3 g of 2.5 | 20.0 g of 2.3 | 19.9 g of 2.3 | 20.0 g of 2.5 | 19.5 g to 2.6 |
| of CO₂ | MPa abs | MPa abs | MPa abs | MPa abs | MPa abs | MPa abs |
| Injection | to 10.4 MPa abs | to 10.5 MPa abs | to 10.3 MPa abs | to 10.3 MPa abs | to 10.5 MPa abs | to 10.7 MPa abs |
| of H₂ | | | | | | |
| Heating | to 50° C. | to 50° C. | to 50° C. | to 50° C. | to 50° C. | to 50° C. |
| Pressure | to 10.0 MPa abs | to 11.5 MPa abs | to 10.5 MPa abs | to 10.2 MPa abs | to 10.6 MPa abs | to 11.6 MPa abs |
| change | | | | | | |
| Reaction | 1 hour | 1 hour | 1 hour | 1 hour | 1 hour | 2 hours |
| time | | | | | | |
| Special | — | — | — | — | — | — |
| feature | | | | | | |
| Upper | 57.5 g | 63.8 g | 60.5 g | 63.3 g | 46.7 g | 48.9 g |
| phase (U1) | 8.0% of 1-propanol | 5.7% of 1-propanol | 3.1% of methanol | 8.4% of methanol | 4.1% of methanol | 0.6% of water |
| | 0.9% of water | 0.5% of water | 96.9% of | 91.6% of | 95.9% of | 4.5% of ethanol |
| | 91.1% of | 93.8% of | tripentylamine | tripentylamine | trihexylamine2 | 94.9% of |
| | trihexylamine | tripentylamine | | | | trihexylamine |
| Lower | 43.6 g | 38.4 g | 40.8 g | 35.9 g | 54.2 g | 61.9 g |
| phase (L1) | 5.9% of formic acid | 6.8% of formic acid | 7.3% of formic acid | 4.5% of formic acid | 7.2% of formic acid | 6.0% of formic acid |
| | 30.3% of 1-propanol | 36.7% of 1-propanol | 36.7% of methanol | 52.1% of methanol | 37.1% of methanol | 12.4% of water |
| | 15.6% of water | 18.2% of water | 18.2% of water | 2.8% of water | 5.5% of water | 36.8% of ethanol |
| | 48.3% of | 38.3% of | 38.3% of | 40.7% of | 50.2% of | 44.8% of |
| | trihexylamine | tripentylamine | tripentylamine | tripentylamine | trihexylamine | trihexylamine |
| k_{Ru} | 1.60 | 2.7 | 4.8 | 14.0 | 1.7 | 3.2 |
| TOF | 252 h⁻¹ | 250 h⁻¹ | 290 h⁻¹ | 343 h⁻¹ | 806 h⁻¹ | 351 h⁻¹ |
| Reaction | 0.54 mol kg⁻¹ h⁻¹ | 0.56 mol kg⁻¹ h⁻¹ | 0.64 mol kg⁻¹ h⁻¹ | 0.35 mol kg⁻¹ h⁻¹ | 0.84 mol kg⁻¹ h⁻¹ | 0.37 mol kg⁻¹ h⁻¹ |
| rate | | | | | | |

### Examples A-7 to A-12

### Hydrogenation Using Diols and Methanol as Solvent

A 100 ml or 250 ml Hastelloy C autoclave equipped with a blade or magnetic stirrer was charged under inert conditions with the tertiary amine (A1), polar solvent and the complex catalyst. The autoclave was subsequently closed and CO₂ was injected at room temperature. H₂ was then injected and the stirrer was heated while stirring (700-1000 rpm). After the given reaction time, the autoclave was cooled and the hydrogenation mixture (H) was depressurized. After the reaction, water was added where applicable to the reaction output and the mixture was stirred for 10 minutes at room temperature. A two-phase hydrogenation mixture (H) was obtained, with the upper phase (U1) being enriched in the tertiary amine (A1) and the complex catalyst and the lower phase (L1) being enriched in the polar solvent and the formic acid-amine adduct (A2) formed. The phases were subsequently separated and the formic acid content of the lower phase (L1) was determined. The total content of formic acid in the formic acid-amine adduct (A2) was determined by potentiometric titration with 0.1 N KOH in MeOH using a “Mettler Toledo DL50” titrator. The parameters and results of the individual experiments are given in Table 1.2 and 1.3

Examples A-7 to A-12 show that, under comparable conditions, higher formic acid concentrations in the lower phase (L1) can be achieved when using methanol/water mixtures as polar solvent compared to diols as polar solvent.

TABLE 1.2

| | Comparative Example | Example A-8 according to | Comparative Example | Example A-10 according to |
|---|---|---|---|---|
| | A-7 | the invention | A-9 | the invention |
| Autoclave | 250 ml | 250 ml | 250 ml | 100 ml |
| Tertiary amine (A1) | trihexylamine 50.0 g | trihexylamine 85.0 g | trihexylamine 50.0 g | trihexylamine 37.5 g |
| Polar solvent | 2-methyl-1,3-propanediol | methanol 25.0 g | 1,4-butanediol 50.0 g | methanol 12.0 g |
| (used) | 50.0 g | water 2.0 g | | water 0.25 g |
| Complex catalyst | [Ru(PnBu₃)₄(H)₂] 100 mg | [Ru(PnOctyl₃)₄(H)₂] 320 mg | [Ru(PnBu₃)₄(H)₂] 100 mg | [Ru(PnOctyl₃)₄(H)₂] 160 mg |
| | 1,2-bis(dicyclohexylphos- | 1,2-bis(dicyclohexyl- | 1,2-bis(dicyclohexyl- | 1,2-bis(dicyclohexyl- |
| | phino)ethane 90 mg | phosphino)ethane 90 mg | phosphino)ethane 90 mg | phosphino)ethane 82 mg |
| Injection of CO₂ | 20.4 g to 3.6 MPa | 26.2 g to 2.8 MPa | 15.5 g to 3.1 MPa | 7.9 g to 2.9 MPa |
| Injection of H₂ | to 11.1 MPa | to 12.0 MPa | to 8.1 MPa | to 8.0 MPa |
| Heating | 50° C. | 50° C. | 50° C. | 50° C. |
| Reaction time | 1 h | 1 h | 1 h | 1 h |
| Formic acid concen- | 7.1% | 8.5% | 2.1% | 8.0% |
| tration in the lower | | | | |
| phase (L1) | | | | |
| Water addition after | — | 2.0 g | — | 1.0 g |
| the reaction | | | | |

TABLE 1.3

| | Example A-11 according | Example A-12 according |
|---|---|---|
| | to the invention | to the invention |
| Autoclave | 250 ml | 250 ml |
| Tertiary | trihexylamine 85.0 g | trihexylamine 85.0 g |
| amine (A1) | | |
| Polar solvent | methanol 15.0 g | methanol 25.0 g |
| (used) | water 2.0 g | water 2.0 g |
| Complex | [Ru(PnOctyl₃)₄(H)₂] | [Ru(PnOctyl₃)₄(H)₂] |
| catalyst | 320 mg | 320 mg |
| | 1,2-bis(dicyclohexylphos- | 1,2-bis(dicyclohexyl- |
| | phino)ethane 80 mg | phosphino)ethane 90 mg |
| Injection | 25.2 g to 2.8 MPa | 25.0 g to 3.0 MPa |
| of CO₂ | | |
| Injection of H₂ | to 19.5 MPa | to 20.0 MPa |
| Heating | 50° C. | 70° C. |
| Reaction time | 10 h | 10 h |
| Formic acid | 12.2% | 9.2% |
| concentration in | | |
| the lower | | |
| phase (L1) | | |
| Water addition | 2.2 | 2.0 g |
| after the reaction | | |

### Examples C-1 to C-11

### According to the Invention (Hydrogenation and Phase Separation, Addition of Water after the Reaction)

A 250 ml Hastelloy C autoclave equipped with a magnetic stirrer bar was charged under inert conditions with tertiary amine (A1), polar solvent and complex catalyst. The autoclave was subsequently closed and CO₂ was injected at room temperature. H₂ was then injected and the reactor was heated while stirring (700 rpm). After the desired reaction time, the autoclave was cooled and the hydrogenation mixture (H) was depressurized. This gave, unless indicated otherwise, after addition of water a two-phase hydrogenation mixture (H), with the upper phase (U1) being enriched in the still free tertiary amine (A1) and the complex catalyst and the lower phase (L1) being enriched in the polar solvent, water and the formic acid-amine adduct (A2) formed. The total content of formic acid in the formic acid-amine adduct (A2) was determined by potentiometric titration with 0.1 N KOH in MeOH using a “Mettler Toledo DL50” titrator. The turnover frequency (=TOF; for the definition of the TOF see: J. F. Hartwig, Organotransition Metal Chemistry, 1^{st} edition, 2010, University Science Books, Sausalito/Calif. p. 545) and the reaction rate were calculated therefrom. The ruthenium content was determined by atomic absorption spectroscopy. The composition of the two phases was determined by gas chromatography and proton NMR spectroscopy. The parameters and results of the individual experiments are shown in Tables 1.4 to 1.7.

In the embodiments in Experiments C-1 to C-9, unfavorable Ru partition coefficients k_{Ru} are present after the reaction. The product phase, viz. stream (3, 13a), 33a), 53a), 73a), 93a)), was therefore subsequently admixed with water to form a two-phase mixture, with the upper phase (U1) comprising mainly tertiary amine (A1) and the alcohol and the lower phase (L1) comprising the formic acid-amine adducts (A2), the alcohol and water and improved Ru partition coefficients between these two phases being established as a result of the addition of water. In addition, very high reaction rates of up to 1.64 mol per kg per hour can be achieved. In the embodiments in comparative experiments for comparison with C3 and C8 (Experiments C-10 and C-11 in Table 1.7), the total amount of water was added in the reaction. It can clearly be seen here that, in the case of the solvents and catalysts used here, the addition of this amount of water in the hydrogenation leads to poorer ruthenium partition coefficients after the reaction and/or lower reaction rates.

TABLE 1.4

| | Example C-1 | | Example C-2 | |
|---|---|---|---|---|
| Tertiary amine (A1) | 75 g of trihexylamine | 50 g of the lower phase | 75 g of tripentylamine | 50 g of the lower phase |
| Polar solvent | 25 g of methanol | from Example C-1 are | 24 g of methanol | from Example C-2 are |
| (used) | | admixed with 6.1 g of | 1 g of methanol | admixed with 7.8 g of |
| Complex catalyst | 0.18 g of [Ru(PⁿBu₃)₄(H)₂] | water. Two phases are | 0.18 g of [Ru(PⁿBu₃)₄(H)₂] | water. Two phases are |
| Injection of CO₂ | 19.9 g to 1.8 MPa abs | formed. | 20.1 g to 2.2 MPa abs | formed. |
| Injection of H₂ | to 9.8 MPa abs | | to 10.2 MPa abs | |
| Heating | to 50° C. | | to 50° C. | |
| Pressure change | to 9.4 MPa abs | | to 10.3 MPa abs | |
| Reaction time | 1 hour | | 1 hour | |
| Special feature | — | | — | |
| Upper phase (U1) | 15.9 g | 18.8 g | 43.9 g | 7.3 g |
| | 12.4% of methanol | 2.8% of methanol | 3.4% of methanol | 1.3% of methanol |
| | 87.6% of trihexylamine | 97.2% of trihexylamine | 96.6% of tripentylamine | 98.7% of tripentylamine |
| Lower phase (L1) | 87.3 g | 36.2 g | 59.2 g | 49.1 g |
| | 5.9% of formic acid | 7.3% of formic acid | 8.7% of formic acid | 8.3% of formic acid |
| | 26.4% of methanol | 16.9% of water | 38.0% of methanol | 17.6% of water |
| | 67.7% of trihexylamine | 35.0% of methanol | 1.7% of water | 38.5% of methanol |
| | | 40.8% of trihexylamine | 51.6% of tripentylamine | 35.6% of tripentylamine |
| K_{Ru} (c_{Ru} in upper phase | 0.3 | 4.0 | 1.1 | 1.7 |
| (U1)/c_{Ru} in lower phase | | | | |
| (L1)) | | | | |
| TOF | 560 h⁻¹ | — | 551 h⁻¹ | — |
| Reaction time | 1.09 mol kg⁻¹ h⁻¹ | — | 1.08 mol kg⁻¹ h⁻¹ | — |

TABLE 1.5

| | Example C-3 | Example C-4 | Example C-5 | Example C-6 |
|---|---|---|---|---|
| Tertiary amine (A1) | 75 g of tripentylamine | 75 g of tripentylamine | 75 g of trihexylamine | 75 g of trihexylamine |
| Polar solvent | 25 g of methanol | 25 g of methanol | 25 g of methanol | 25 g of methanol |
| Complex catalyst | 0.16 g of | 0.33 g of | 0.16 g of | 0.32 g of |
| | [Ru(PnOct3)4(H)2] | [Ru(PnOct3)4(H)2] | [Ru(PnOctyl₃)₄(H)₂] | [Ru(PnOctyl₃)₄(H)₂], |
| | | | | 0.17 g of 1,2- |
| | | | | bis(dicyclohexylphosphino) |
| | | | | ethane, 0.15 g of PnOctyl₃ |
| Injection of CO₂ | 19.9 g to 2.1 MPa abs | 20.0 g to 2.0 MPa abs | to 1.9 MPa abs | to 2.0 MPa abs |
| Injection of H₂ | to 10.0 MPa abs | to 10.0 MPa abs | to 9.9 MPa abs | to 12.0 MPa abs |
| Heating | to 50° C. | to 50° C. | 70° C. | 50° C. |
| Pressure change | to 10.6 MPa abs | to 10.8 MPa abs | to 11.1 MPa abs | to 12.4 MPa abs |
| Reaction time | 1 hour | 1 hour | 1 hour | 1 hour |
| Special feature | addition of 5 g of water | addition of 3 g of water | addition of 5 g of water | addition of 5 g of water |
| | after the reaction | after the reaction | after the reaction | after the reaction |
| Upper phase (U1) | 55.5 g | 40.5 g | 55.2 g | 25.9 g |
| | 3.1% of methanol | VH10-44 | | |
| | 96.9% of tripentylamine | | | |
| Lower phase (L1) | 45.5 g | 61.5 g | 43.4 g | 78.0 g |
| | 6.1% of formic acid | 7.0% of formic acid | 5.4% of formic acid | 8.5% of formic acid |
| | 51.2% of methanol | 35.9% of methanol | | |
| | 11.0% of water | 4.9% of water | | |
| | 31.7% of tripentylamine | 52.2% of trihexylamine | | |
| K_{Ru} | 10.9 | 41.0 | 100 | 19.4 |
| TOF | 586 h−1 | 447 h−1 | 492 h⁻¹ | 696 |
| Reaction rate | 0.60 mol kg−1 h−1 | 0.91 mol kg−1 h−1 | 0.52 mol kg⁻¹ h⁻¹ | 1.38 mol kg⁻¹ h⁻¹ |

TABLE 1.6

| | Example C-7 | Example C-8 | Example C-9 |
|---|---|---|---|
| Tertiary amine (A1) | 75 g of trihexylamine | 75 g of trihexylamine | 75 g of trihexylamine |
| Polar solvent | 25 g of methanol | 25 g of methanol | 25 g of ethanol |
| Complex catalyst | 0.11 g [Ru(COD)Cl₂]₂, | 0.32 g of [Ru(PnOctyl₃)₄(H)₂], | 0.18 g of [Ru(PⁿOct₃)₄(H)₂] |
| | 0.17 g of 1,2- | 0.08 g of 1,2- | |
| | bis(dicyclohexylphos- | bis(dicyclohexylphosphino)- | |
| | phino)ethane | ethane | |
| | 0.15 g of PnOct₃ | | |
| Injection of CO₂ | to 1.6 MPa abs | to 1.7 MPa abs | 20.0 g to 2.2 MPa abs |
| Injection of H₂ | to 12.0 MPa abs | to 9.7 MPa abs | to 10.2 MPa abs |
| Heating | 50° C. | 50° C. | to 50° C. |
| Pressure change | to 12.1 MPa abs | to 9.5 MPa abs | to 11.1 MPa abs |
| Reaction time | 1 hour | 2 hours | 1 hour |
| Special feature | addition of 5 g of water | addition of 5 g of water after | Single-phase reaction output; |
| | after the reaction | the reaction | addition of 5 g of water after |
| | | | the reaction, resulting in |
| | | | formation of two phases |
| Upper phase (U1) | 16.6 g | 26.7 g | 66.3 g |
| | | | 8.8% of ethanol |
| | | | 0.8% of water |
| | | | 90.4% of trihexylamine |
| Lower phase (L1) | 88.1 g | 74.0 g | 29.6 g |
| | 9.0% of formic acid | 8.3% of formic acid | (2.7% of formic acid, 20.9% of |
| | | | water, 64.4% of ethanol, 12% |
| | | | of trihexylamine) |
| K_{Ru} | 12.0 | 14 | 22.5 |
| TOF | 435 h⁻¹ | 335 h⁻¹ | 152 h⁻¹ |
| Reaction rate | 1.64 mol kg⁻¹ h⁻¹ | 1.33 mol kg⁻¹ h⁻¹ | 0.18 mol kg⁻¹ h⁻¹ |

TABLE 1.7
Addition of water in the reaction

| | Example C-10 (comparative | Example C-11 (comparative |
|---|---|---|
| | experiment for C8) | experiment for C3) |
| Tertiary amine (A1) | 75 g of trihexylamine | 75 g of tripentylamine |
| Polar solvent | 25 g of methanol | 25 g of methanol |
| (used) | 5.0 g of water | 5.0 g of water |
| Catalyst | 0.32 g of [Ru(PnOctyl₃)₄(H)₂], | 0.16 g of [Ru(PnOctyl₃)₄(H)₂] |
| | 0.08 g of 1,2- | |
| | bis(dicyclohexylphosphino)ethane | |
| Injection of CO₂ | 20.0 g to 2.5 MPa abs | 20.0 g to 2.1 MPa abs |
| Injection of H₂ | to 10.6 MPa abs | to 10.1 MPa abs |
| Heating | to 50° C. | to 50° C. |
| Pressure change | to 11.0 MPa abs | to 11.3 MPa abs |
| Reaction time | 1 hour | 1 hour |
| Special feature | water is added before the reaction | water is added before the |
| | | reaction |
| Upper phase (U1) | 64.3 g | 66.6 g |
| Lower phase (L1) | 41.7 g | 37.6 g |
| | 4.7% of formic acid | 2.4% of formic acid |
| K_{Ru} (c_{Ru} in upper phase | 1.3 | 32.5 |
| (U1)/c_{Ru} in lower phase | | |
| (L1)) | | |
| TOF | 394 h⁻¹ | 187 h⁻¹ |
| Reaction rate | 0.4 mol kg⁻¹ h⁻¹ | 0.19 mol kg⁻¹ h⁻¹ |

### Examples D1-D4

### Extraction of the Complex Catalyst

A 100 ml Hastelloy C autoclave equipped with a blade stirrer was charged under inert conditions with the tertiary amine (A1), polar solvent and the complex catalyst. The autoclave was subsequently closed and CO₂ was injected at room temperature. H₂ was then injected and the reactor was heated while stirring (1000 rpm). After the given reaction time, the autoclave was cooled and the hydrogenation mixture (H) was depressurized. After the reaction, water was added to the hydrogenation mixture and the mixture was stirred at room temperature for 10 minutes. This gave a two-phase hydrogenation mixture (H), with the upper phase (U1) being enriched in the still free tertiary amine (A1) and the homogeneous catalyst, and the lower phase (L1) being enriched in the polar solvent and the formic acid-amine adduct (A2) formed. The lower phase (L1) was separated off and treated three times under inert conditions with the same amount (mass of tertiary amine corresponds to the mass of the lower phase) of fresh tertiary amine (stir at room temperature for 10 minutes and subsequently separate the phases). The total content of formic acid in the formic acid-amine adduct was determined by potentiometric titration with 0.1 N KOH in MeOH using a “Mettler Toledo DL50” titrator. The ruthenium content was determined by AAS. The parameters and results of the individual experiments are shown in Table 1.8.

Examples D-1 to D-4 show that the ruthenium content of the product phase (raffinate R2) can be reduced to less than one ppm of ruthenium by varying the catalyst and the amount of water added in the formation of formic acid.

TABLE 1.8

| | Example D-1 | Example D-2 | Example D-3 | Example D-4 |
|---|---|---|---|---|
| Tertiary amine (A1) | 37.5 g of trihexylamine | 37.5 g of trihexylamine | 37.5 g of trihexylamine | 37.5 g of trihexylamine |
| Polar solvent | 12.0 g of methanol | 12.0 g of methanol | 12.0 g of methanol | 12.0 g of methanol |
| (used) | | | | 0.5 g of water |
| Complex catalyst | 0.16 g of | 0.16 g of [Ru(PnOctyl₃)₄(H)₂] | 0.16 g of [Ru(PnOctyl₃)₄(H)₂] | 0.1 g of [Ru(PnButyl₃)₄(H)₂] |
| | [Ru(PnOctyl₃)₄(H)₂] | | | |
| Injection of CO₂ | to 1.7 MPa abs | to 1.6 MPa abs | to 1.8 MPa abs | to 1.7 MPa abs |
| Injection of H₂ | to 8.0 MPa abs | to 8.0 MPa | to 8.0 MPa | to 8.0 MPa |
| Heating | 50° C. | 50° C. | 50° C. | 50° C. |
| Reaction time | 1.5 hours | 1.5 hours | 16 hours | 1.5 hours |
| Water addition after the | 2.5 g | 4.7 g | 2.5 g | 0.8 g |
| reaction | | | | |
| Upper phase (U1) | 26.3 g | 27.4 g | 23.2 g | 17.5 g |
| Lower phase (L1) | 24.7 g | 25.5 g | 28.1 g | 28.9 g |
| | 6.6% of formic acid | 5.9% of formic acid | 6.8% of formic acid | 7.4% of formic acid |
| c_{Ru} in upper phase (U1) | 350 ppm | 280 ppm | 370 ppm | 200 ppm |
| after the reaction and | | | | |
| addition of water | | | | |
| c_{Ru} in lower phase (L1) after | 4 ppm | 2 ppm | <1 ppm | 43 ppm |
| extraction (raffinate (R2)) | | | | |

### Examples E1-E5

### Reuse of the Catalyst and Catalyst Extraction)

A 100 ml Hastelloy C autoclave equipped with a blade stirrer was charged under inert conditions with the tertiary amine (A1), polar solvent and the complex catalyst. The autoclave was subsequently closed and CO₂ was injected at room temperature. H₂ was then injected and the reactor was heated while stirring (1000 rpm). After the reaction time, the autoclave was cooled and the hydrogenation mixture (H) was depressurized. After the reaction, water was added to the reaction output and the mixture was stirred at room temperature for 10 minutes. This gave a two-phase hydrogenation mixture (H), with the upper phase (U1) being enriched in the still free tertiary amine (A1) and the complex catalyst and the lower phase (L1) being enriched in the polar solvent and the formic acid-amine adduct (A2) formed. The phases were subsequently separated and the formic acid content of the lower phase (L1) and also the ruthenium content of both phases were determined by the methods described below. The upper phase (U1) comprising ruthenium catalyst was then made up to 37.5 g with fresh tertiary amine (A1) and reused for the hydrogenation of CO₂ using the same solvent under the same reaction conditions as before. After the reaction was complete and water had been added, the lower phase (L1) was separated off and admixed three times under inert conditions with the same amount (mass of amine corresponds to the mass of the lower phase) of fresh tertiary amine (A1) (stir at room temperature for 10 minutes and subsequently separate the phases) to extract the catalyst. The total content of formic acid in the formic acid-amine adduct (A2) was determined by potentiometric titration with 0.1 N KOH in MeOH using a “Mettler Toledo DL50” titrator. The ruthenium content was determined by AAS. The parameters and results of the individual experiments are shown in Tables 1.9 to 1.0.

Examples E-1 to E-5 show that varying the catalyst, the amount of water added (both before and after the reaction) and the reaction conditions allows the active catalyst to be reused for the hydrogenation of CO₂ and allows the ruthenium content of the product phase to be reduced to as low as 2 ppm by means of only a single extraction.

TABLE 1.9

| | Example E-1a (first | Example E-1b (reuse of the | Example E-2a (first | Example E-2b (reuse of the | | Example E-3b (reuse of the |
|---|---|---|---|---|---|---|
| | hydrogenation) | catalyst and extraction) | hydrogenation) | catalyst and extraction) | Example E-3a (first hydrogenation) | catalyst and extraction) |
| Tertiary amine (A1) | 37.5 g of trihexylamine | Upper phase from E-1a made up | 37.5 g of trihexylamine | Upper phase from E-2a | 37.5 g of trihexylamine | upper phase from E-3a made |
| | | to 37.5 g with fresh trihexylamine | | made up to 37.5 g with fresh | | up to 37.5 g with fresh |
| | | | | trihexylamine | | trihexylamine |
| Polar solvent | 12.0 g of methanol | 12.0 g of methanol | 12.0 g of methanol | 12.0 g of methanol | 12.0 g of methanol | |
| (used) | | | 0.5 g of water | 0.5 g of water | | |
| Complex catalyst | 0.16 g of | Upper phase from E-1a | 0.16 g of | Upper phase from E-2a | 0.16 g of [Ru(PnOctyl₃)₄(H)₂] | upper phase from E-3a |
| | [Ru(PnOctyl₃)₄(H)₂], | | [Ru(PnOctyl₃)₄(H)₂], | | | |
| | 0.08 g of 1,2-bis(dicyclo- | | 0.08 g of 1,2-bis(dicyclo- | | | |
| | hexylphosphino)ethane | | hexylphosphino)ethane | | | |
| Injection of CO₂ | to 1.8 MPa abs | to 1.6 MPa abs | to 1.8 MPa abs | to 1.7 MPa abs | to 1.7 MPa abs | to 1.7 MPa abs |
| Injection of H₂ | to 8.0 MPa abs | to 8.0 MPa | to 8.0 MPa abs | to 8.0 MPa abs | to 8.0 MPa abs | to 8.0 MPa abs |
| Heating | 70° C. | 70° C. | 70° C. | 70° C. | 50° C. | 50° C. |
| Reaction time | 16 hours | 1.5 hours | 16 hours | 1.5 hours | 1.5 hours | 1.5 hours |
| Water addition after the | 1.0 g | 1.0 g | 1.0 g | 1.0 g | 2.5 g | 1.0 g |
| reaction | | | | | | |
| Upper phase (U1) | 19.9 g | 24.7 g | 19.7 g | 24.0 g | 23.8 g | 28.6 g |
| Lower phase (L1) | 30.8 g | 24.4 g | 31.1 g | 26.8 g | 26.9 g | 20.6 g |
| | 6.8% of formic acid | 6.0% of formic acid | 7.1% of formic acid | 6.4% of formic acid | 6.2% of formic acid | 4.8% of formic acid |
| c_{Ru} in upper phase | 205 ppm | 135 ppm | 250 ppm | 175 ppm | 400 ppm | 310 ppm |
| (U1) after the reaction | | | | | | |
| and addition of water | | | | | | |
| c_{Ru} in lower phase (L1) | 145 ppm | — | 125 ppm | — | 4 ppm | — |
| after the reaction and | | | | | | |
| addition of water | | | | | | |
| c_{Ru} in lower phase after | — | 4 ppm | — | 4 ppm | — | 2 ppm |
| extraction (raffinate | | | | | | |
| (R2)) | | | | | | |

TABLE 1.10

| | | Example E-4b (reuse | | Example E-5b (reuse | Example E-5c (reuse |
|---|---|---|---|---|---|
| | Example E-4a (first | of the catalyst and | Example E-5a (first | of the catalyst and | of the catalyst and |
| | hydrogenation) | extraction) | hydrogenation) | extraction) | extraction) |
| Tertiary amine (A1) | 37.5 g of trihexylamine | upper phase from E-4a | 37.5 g of trihexylamine | upper phase from E-5a | upper phase from E-5b |
| | | made up to 37.5 g with | | made up to 37.5 g with | made up to 37.5 g with |
| | | fresh trihexylamine | | fresh trihexylamine | fresh trihexylamine |
| Polar solvent | 12.0 g of methanol | 12.0 g of methanol | 12.0 g of methanol | 12.0 g of methanol | 12.0 g of methanol |
| (used) | | | 0.5 g of water | | |
| Complex catalyst | 0.16 g of | upper phase from E-4a | 0.16 g of | upper phase from E-5a | upper phase from E5b |
| | [Ru(PnOctyl₃)₄(H)₂], | | [Ru(PnOctyl₃)₄(H)₂], | | |
| | 0.08 g of 1,2- | | 0.08 g of 1,2- | | |
| | bis(dicyclohexylphos- | | bis(dicyclohexylphos- | | |
| | phino)ethane | | phino)ethane | | |
| Injection of CO₂ | to 1.7 MPa abs | to 1.8 MPa abs | to 1.5 MPa abs | to 1.6 MPa abs | to 1.6 MPa abs |
| Injection of H₂ | to 8.0 MPa abs | to 8.0 MPa | to 8.0 MPa abs | to 8.0 MPa | to 8.0 MPa |
| Heating | 70° C. | 70° C. | 70° C. | 70° C. | 70° C. |
| Reaction time | 16 hours | 1.5 hours | 16 hours | 1.5 hours | 1.5 hours |
| Water addition after | 1.0 g | 1.0 g | 1.0 g | 1.0 g | 1.0 g |
| the reaction | | | | | |
| Upper phase (U1) | 20.4 g | 27.3 g | 19.7 g | 27.8 g | 25.6 g |
| Lower phase (L1) | 29.8 g | 22.3 g | 31.6 g | 22.6 g | 24.4 g |
| | 6.7% of formic acid | 5.7% of formic acid | 7.0% of formic acid | 6.1% of formic acid | 6.1% of formic acid |
| c_{Ru} in upper phase | 215 ppm | 150 ppm | 235 ppm | 155 ppm | 125 ppm |
| (U1) after the reaction | | | | | |
| and addition of water | | | | | |
| c_{Ru} in lower phase (L1) | 145 ppm | 14 ppm | 110 ppm | 11 ppm | — |
| after the reaction and | | | | | |
| addition of water | | | | | |
| c_{Ru} in lower phase | — | — | — | — | 3 ppm |
| after extraction | | | | | |
| (raffinate (R2)) | | | | | |

### Examples F1-F4

### Thermal Separation of the Polar Solvent; Step (c)

Alcohol and water are distilled off from the product phase (comprises the formic acid-amine adduct; corresponding to lower phase (L1), raffinate (R1) or raffinate (R2)) under reduced pressure by means of a rotary evaporator. A two-phase mixture (trialkylamine and formic acid-amine adduct phase; corresponding to bottoms mixture (S1)), is formed at the bottom, and the two phases are separated and the formic acid content of the lower phase (L2) was determined by potentiometric titration with 0.1 N KOH in MeOH using a “Mettler Toledo DL50” titrator. The amine and alcohol content is determined by gas chromatography. The parameters and results of the individual experiments are shown in Table 1.1.

Examples F-1 to F-4 show that various polar solvents can be separated off under mild conditions from the product phase (lower phase (L1); raffinate (R1) or raffinate (R2)) in the process of the invention, giving a lower phase (L2) which is relatively rich in formic acid and an upper phase (U2) comprising predominantly tertiary amine.

TABLE 1.22

| | Example F-1 | Example F-2 | Example F-3 | Example F-4 |
|---|---|---|---|---|
| Feed mixture | 18.7 g | 19.3 g | 81.8 g | 88.6 g |
| (% by weight) | 7.2% of formic acid | 5.8% of formic acid | 7.3% of formic acid | 9.2% of formic acid |
| | 26.4% of 1-propanol | 22.8% of 2-propanol | 41.3% of methanol | 31.4% of ethanol |
| | 15.5% of water | 4.1% of water | 15.4% of water | 11.3% of water |
| | 48.3% of | 67.2% of | 35.9% of | 48.1% of |
| | trihexylamine | trihexylamine | tripentylamine | tripentylamine |
| Formic acid:amine in | 1:1.2 | 1:2.0 | 1:1 | 1:1.1 |
| feed mixture | | | | |
| Pressure | 20 mbar | 20 mbar | 200 mbar | 200 mbar |
| Temperature | 50° C. | 50° C. | 100° C. | 110° C. |
| Formic acid content of | 16.4% | 18.0% | 23.7% | 22.7% |
| lower phase after | | | | |
| distillation (% by weight) | | | | |
| Formic acid:amine in | 1:0.76 | 1:0.78 | 1:0.6 | 1:0.56 |
| lower phase after | | | | |
| distillation (molar ratio) | | | | |
| Recovery of formic acid | 95.3% | 93.7% | 90.4% | 95.2% |
| after distillation | | | | |

### Examples G1 and G2

### Thermal Separation of the Polar Solvent from the Trialkylamine/Solvent/Formic Acid Mixtures and Dissociation of the Formic Acid-Amine Adduct

Alcohol and water are distilled off from the product phase (comprises the formic acid-amine adduct; corresponding to lower phase (L1), raffinate (R1) or raffinate (R2)) under reduced pressure by means of a rotary evaporator. A two-phase mixture (trialkylamine and formic acid-amine adduct phase; bottoms mixture (S1)) is formed at the bottom and the two phases are separated. The composition of the distillate (comprising the major part of the methanol and of the water; distillate (D1)), the upper phase (comprising the free trialkylamine; upper phase (U2)) and the lower phase (comprising the formic acid-amine adduct; lower phase (L2)) was determined by gas chromatography and by potentiometric titration of the formic acid against 0.1 N KOH in MeOH using a “Mettler Toledo DL50” titrator. The formic acid is then thermally split off from the tertiary amine (A2) in the lower phase (L2) from the first step via a 10 cm Vigreux column in a vacuum distillation apparatus. After all the formic acid has been split off, a single-phase bottom fraction (S2) comprising the pure tertiary amine (A2) is obtained and can be used for extraction of the catalyst and recirculation to the hydrogenation. The formic acid and residual water are present in the distillate (D2). The composition of the bottoms (S2) and of the distillate was determined by gas chromatography and by potentiometric titration of the formic acid against 0.1 N KOH in MeOH using a “Mettler Toledo DL50” titrator. The parameters and results of the individual experiments are shown in Table 1.12.

Examples G-1 and G-2 show that various polar solvents can be separated off from the product phase under mild conditions in the process of the invention, giving a lower phase (L3) which is relatively rich in formic acid and an upper phase (U3) comprising predominantly tertiary amine (A1). The formic acid can then be split off from the tertiary amine (A1) in this lower phase (L3) which is relatively rich in formic acid at relatively high temperatures, leaving the free tertiary amine (A1). The formic acid which has been obtained in this way still comprises some water which can be separated off from the formic acid by means of a column having a relatively high separating power. The tertiary amine (A1) obtained both in the removal of the solvent and in the thermal dissociation can be used for extracting the catalyst.

TABLE 1.12

| | | Example G-1b | | |
|---|---|---|---|---|
| | Example G-1a | (dissociation of the | Example G-2a | Example G-2b |
| | (removal of the polar | formic acid-amine | (removal of the polar | (dissociation of the formic |
| | solvent) | adduct) | solvent) | acid-amine adduct) |
| Feed mixture | 199.8 g | lower phase from G-1a | 199.8 g | lower phase from G-2a |
| (% by weight) | 8.9% of formic acid | | 7.8% of formic acid | |
| | 28.4% of methanol | | 33.0% of methanol | |
| | 5.6% of water | | 15.1% of water | |
| | 57.1% of trihexylamine | | 44.0% of trihexylamine | |
| Formic acid:amine in | 1:1.1 | 1:0.64 | 1:1 | 1:0.89 |
| feed mixture | | | | |
| Pressure | 200 mbar | 90 mbar | 200 mbar | 90 mbar |
| Temperature | 120° C. | 153° C. | 120° C. | 153° C. |
| Lower phase in the | 79.8 g | 63.6 g | 69.4 g | 55.5 g |
| bottoms after distillation | 22.1% of formic acid | 100% of trihexylamine | 14.9% of formic acid | 99.7% of trihexylamine |
| (% by weight) | 1.5% of water | | 6.9% of water | 0.3% of water |
| | 76.4% of trihexylamine | | 78.2% of trihexylamine | |
| Upper phase in the | 50.5 g | single-phase | 32.7 g | single-phase |
| bottoms after distillation | 100% of trihexylamine | | 99.7% of trihexylamine | |
| | | | 0.3% of water | |
| Distillate | 66.6 g | 14.9 g | 93.1 g | 12.9 g |
| | 0.3% of formic acid | 92.1% of formic acid | 70.1% of methanol | 85.0% of formic acid |
| | 81.2% of methanol | 7.9% of water | 29.9% of water | 15% of water |
| | 18.5% of water | | | |

### Examples H1 to H4

### Inhibition of the Complex Catalyst During the Solvent Removal and The Thermal Dissociation; Steps (c) and (e)

The decomposition experiments for the solvent removal and the dissociation of the formic acid-amine adduct (A2) were carried out in 250 ml three-neck glass flasks provided with reflux condenser and argon blanketing. Inhibition of the complex catalyst by means of CO was carried out during the experiment by means of a metal frit through which CO was bubbled into the solution (5-6 l of CO/h). The reaction mixture was boiled under reflux. The samples for determining the phase ratio and the formic acid concentration were taken through a septum by means of a syringe during the reaction. The formic acid concentration was determined by potentiometric titration against 0.1 N KOH in MeOH using a “Mettler Toledo DL50” titrator.

Synthesis of the catalyst stock solution (CS1) for inhibition experiments: in air, 3.15 g of [Ru(COD)Cl₂] are placed in a 1.2 l Hastelloy autoclave and 150 g of trihexylamine (THA) are added. The autoclave is subsequently closed, tested for freedom from leaks using N₂ and flushed with N₂. A mixture of 4.66 g of 1,2-dicylohexylphosphinoethane, 8.19 g of trioctylphosphane, 567 g of THA, 57.25 g of MeOH and 6.3 g of water is subsequently sucked into the autoclave under argon by application of a vacuum. The autoclave is then heated to 70° C. while stirring and 160 g of CO₂ are injected. H₂ is injected to 120 bar and the pressure is maintained at 120 bar during the reaction by injection of further H₂. After 4 hours, the autoclave is cooled to RT and depressurized to atmospheric pressure. 20 g of water are added to the reaction output while stirring to give a two-phase mixture. The phases are separated. This gives 501 g of an upper phase, which comprises trihexylamine and the active catalyst (1900 ppm of Ru) and 156 g of lower phase (680 ppm of Ru), which is discharged. The upper phase comprises 83.5% of the ruthenium used and is subsequently used as catalyst stock solution (CS1) for the inhibition experiments.

### Inhibition in the Thermal Dissociation of the Formic Acid-Amine Adduct (A2); Step (e): Experiment H-1

200 ppm of Ru in the form of [Ru(PnOct₃)₄(H)₂], 20 mg of dcpe (1,2-dicyclo-hexylphosphinoethane) and 80 g of the formic acid-trihexylamine adduct (A3) (N(Hex)₃:FA=1:1.5; 20.4% by weight of formic acid (FA)) are in each case placed in a 250 ml glass flask and heated to 130° C. A first experiment in which CO is passed through (“passage of CO”) is carried out. A second experiment in which no CO is added (“without inhibitor”) is carried out. The formic acid decomposition (FA-D [%]) and the formic acid concentration (FA[%]) are determined by sampling and titration. The results of the first and second experiments are shown in graphical form in FIG. 7.

Example H-1 shows that in the process of the invention, the decomposition of formic acid due to residues of the complex catalyst under the conditions of the thermal dissociation of the formic acid-amine adducts can be largely suppressed by addition of CO.

### Inhibition in the Solvent Removal (Step (c)): Experiment H-2

11.1 g of the catalyst stock solution CS1, 53.9 g of trihexylamine, 25 g of methanol, 2 g of water and 7.8 g of formic acid are placed in a 250 ml glass flask and heated under reflux. A first experiment in which CO is passed through (“passage of CO”) is carried out. A second experiment in which no CO is added (“without inhibitor”) is carried out. The formic acid decomposition and the formic acid concentration (FA[%]) are determined by sampling and titration. The results of the first and second experiments are shown in graphical form in FIG. 8.

Example H-2 shows that in the process of the invention, the decomposition of formic acid due to residues of the catalyst under the conditions of the solvent removal (step (c)) can be largely suppressed by addition of CO.

### Inhibition in the Solvent Removal (Step (c)) and Catalyst Recirculation: Experiment H-3

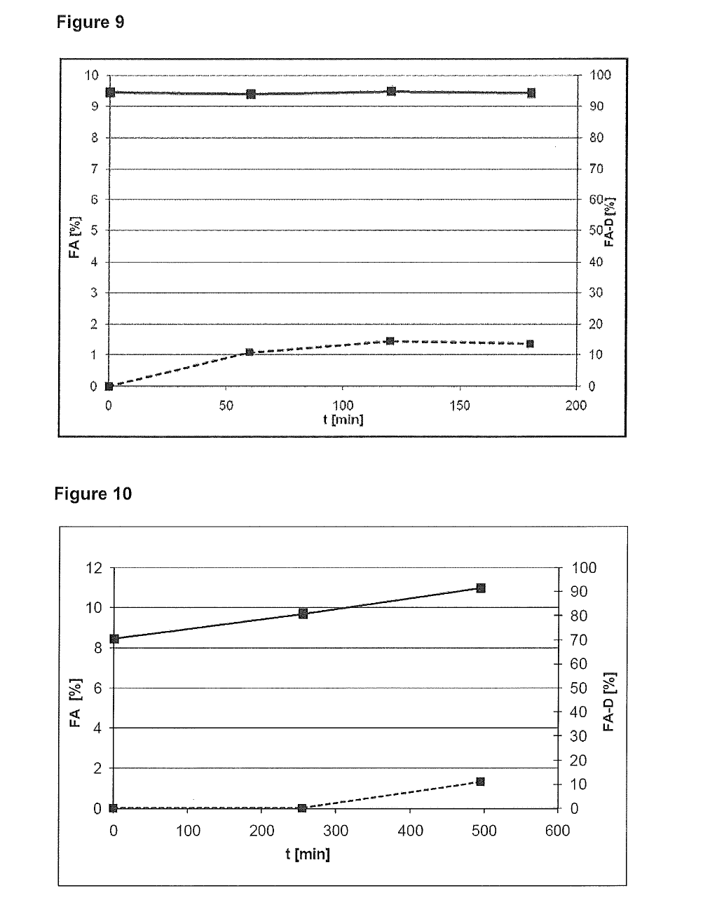

11.1 g of the catalyst stock solution CS1, 53.9 g of trihexylamine, 25 g of methanol, 2 g of water and 7.8 g of formic acid are placed in a 250 ml glass flask and heated under reflux. A first experiment in which CO is passed through (“passage of CO”) is carried out. A second experiment in which no CO is added (“without inhibitor”) is carried out. The formic acid decomposition and the formic acid concentration (FA[%]) are determined by sampling and titration. The results of the first and second experiments are shown in graphical form in FIG. 9.

The amine phase, which comprises the major part of the inhibited complex catalyst, is subsequently separated off and used in the CO₂ hydrogenation. For this purpose, 37.5 g of the amine phase from the inhibition experiment, 12.5 g of methanol and 1 g of water are placed in a 100 ml HC autoclave. The autoclave is made inert by means of N₂ and 10 g of CO₂ are injected (22 bar). H₂ is subsequently injected at 70° C. to 80 bar and the pressure is maintained at 80 bar over the reaction time by injection of further H₂. The concentration of formic acid in the product phase is 1.6% after 2 hours and 4.3% after 16 hours.

Example H-3 shows that in the process of the invention, the inhibited complex catalyst can be reconverted into the active form under the hydrogenation conditions.

### Inhibition in the Solvent Removal (Step (c)) and Thermal Reactivation of the Catalyst: Experiment H-4

11.1 g of the catalyst stock solution CS1, 53.9 g of trihexylamine, 25 g of methanol, 2 g of water and 7.8 g of formic acid are placed in a 250 ml glass flask and heated under reflux and CO is passed through the reaction mixture (“passage of CO”). The formic acid decomposition and the formic acid concentration (FA[%]) are determined by sampling and titration. The results of the first and second experiments are shown in graphical form in FIG. 10.

This reaction mixture is subsequently boiled under reflux for a further 10 hours without addition of CO in order to reactivate the inhibited complex catalyst. After this time, the formic acid has been completely decomposed. The amine phase, which comprises the major part of the complex catalyst, is then separated off and reused in the CO₂ hydrogenation. For this purpose, 37.5 g of the amine phase from the inhibition experiment, 12.5 g of methanol and 1 g of water are placed in a 100 ml HC autoclave. The autoclave is made inert by means of N₂ and 10 g of CO₂ are injected (21 bar). H₂ is subsequently injected at 70° C. to 80 bar and the pressure is maintained at 80 bar over the reaction time by the injection of further H₂. The concentration of formic acid in the product phase is 5.5% after one hour. The partition coefficient of the ruthenium between the amine phase and product phase is 18.8.

Example H-4 shows that in the process of the invention, the inhibited complex catalyst can be reconverted into the active form by thermal treatment without CO under the hydrogenation conditions and is then significantly faster in the hydrogenation and also that the complex catalyst is preferentially present in the amine phase even after inhibition and reactivation.

1-14. (canceled)

15. A process for preparing formic acid, which comprises the steps
(a) homogeneously catalyzed reaction of a reaction mixture (Rg) comprising carbon dioxide, hydrogen, at least one polar solvent selected from the group consisting of methanol, ethanol, 1-propanol, 2-propanol, 1-butanol, 2-butanol, 2-methyl-1-propanol and water and also at least one tertiary amine of the general formula (A1)
NR¹R²R³ (A1),
where
R¹, R² and R³ are each, independently of one another, an unbranched or branched, acyclic or cyclic, aliphatic, araliphatic or aromatic radical having in each case from 1 to 16 carbon atoms, where individual carbon atoms may, independently of one another, also be replaced by a heterogroup selected from among the groups —O— and >N— and two or all three radicals can also be joined to one another to form a chain comprising at least four atoms,
in the presence of at least one complex catalyst comprising at least one element selected from groups 8, 9 and 10 of the Periodic Table,
in a hydrogenation reactor
to give, optionally after addition of water, a two-phase hydrogenation mixture (H) comprising
an upper phase (U1), which comprises the at least one complex catalyst and the at least one tertiary amine (A1) and
a lower phase (L1) which comprises the at least one polar solvent, residues of the at least one complex catalyst and also at least one formic acid-amine adduct of the general formula (A2),
NR¹R²R³*xᵢHCOOH (A2),
where
xᵢ is in the range from 0.4 to 5 and
R¹, R² and R³ are as defined above,
(b) work-up of the hydrogenation mixture (H) obtained in step (a) according to one of the following steps
(b1) phase separation of the hydrogenation mixture (H) obtained in step (a) into the upper phase (U1) and the lower phase (L1) in a first phase separation apparatus
or
(b2) extraction of the at least one complex catalyst from the hydrogenation mixture (H) obtained in step (a) by means of an extractant comprising the at least one tertiary amine (A1) in an extraction unit to give
a raffinate (R1) comprising the at least one formic acid-amine adduct (A2) and the at least one polar solvent and
an extract (E1) comprising the at least one tertiary amine (A1) and the at least one complex catalyst
or
(b3) phase separation of the hydrogenation mixture (H) obtained in step (a) into the upper phase (U1) and the lower phase (L1) in a first phase separation apparatus and extraction of the residues of the at least one complex catalyst from the lower phase (L1) by means of an extractant comprising the at least one tertiary amine (A1) in an extraction unit to give
a raffinate (R2) comprising the at least one formic acid-amine adduct (A2) and the at least one polar solvent and
an extract (E2) comprising the at least one tertiary amine (A1) and the residues of the at least one complex catalyst,
(c) separating the at least one polar solvent from the lower phase (L1), from the raffinate (R1) or from the raffinate (R2) in a first distillation apparatus to give
a distillate (D1) comprising the at least one polar solvent, which is recirculated to the hydrogenation reactor in step (a), and
a two-phase bottoms mixture (S1) comprising
an upper phase (U2) which comprises the at least one tertiary amine (A1) and a lower phase (L2) which comprises the at least one formic acid-amine adduct (A2),
(d) optionally work-up of the bottoms mixture (S1) obtained in step (c) by phase separation in a second phase separation apparatus to give the upper phase (U2) and the lower phase (L2),
(e) dissociating the at least one formic acid-amine adduct (A2) comprised in the bottoms mixture (S1) or optionally in the lower phase (L2) in a thermal dissociation unit to give the at least one tertiary amine (A1), which is recirculated to the hydrogenation reactor in step (a), and formic acid, which is discharged from the thermal dissociation unit,
wherein carbon monoxide is added to the lower phase (L1), the raffinate (R1) or the raffinate (R2) directly before and/or during step (c)
and/or
carbon monoxide is added to the bottoms mixture (S1) or optionally to the lower phase (L2) directly before and/or during step (e).

16. The process according to claim 15, wherein the hydrogenation mixture (H), obtained in step (a) is worked up further according to step (b1) and the upper phase (U1) is recirculated to the hydrogenation reactor in step (a) and the lower phase (L1) is fed to the first distillation apparatus in step (c).

17. The process according to claim 15, wherein the hydrogenation mixture (H) obtained in step (a) is worked up further according to step (b2), with the at least one tertiary amine (A1) obtained in the thermal dissociation unit in step (e) being used as extractant and the extract (E1) being recirculated to the hydrogenation reactor in step (a) and the raffinate (R1) being fed to the first distillation apparatus in step (c).

18. The process according to claim 15, wherein the hydrogenation mixture (H) obtained in step (a) is worked up further according to step (b3), with the at least one tertiary amine (A1) obtained in the thermal dissociation unit in step (e) being used as extractant and the extract (E2) being recirculated to the hydrogenation reactor in step (a) and the raffinate (R2) being fed to the first distillation apparatus in step (c).

19. The process according to claim 15, wherein the thermal dissociation unit comprises a second distillation apparatus and a third phase separation apparatus and the dissociation of the formic acid-amine adduct (A2) is carried out in the second distillation apparatus to give
a distillate (D2) comprising formic acid which is discharged from the second distillation apparatus and
a two-phase bottoms mixture (S2) comprising
an upper phase (U3) which comprises the at least one tertiary amine (A1), inhibited complex catalyst and free ligands, and a lower phase (L3) which comprises the at least one formic acid-amine adduct (A2).

20. The process according to claim 19, wherein the bottoms mixture (S2) obtained in the second distillation apparatus is separated into the upper phase (U3) and the lower phase (L3) in the third phase separation apparatus of the thermal dissociation unit and the upper phase (U3) is recirculated to the hydrogenation reactor in step (a) and the lower phase (L3) is recirculated to the second distillation apparatus of the thermal dissociation unit.

21. The process according to claim 20, wherein the upper phase (U3) is recirculated to the extraction unit in step (b2) or (b3).

22. The process according to claim 15, wherein the first bottoms mixture (S1) obtained in step (c) or optionally the lower phase (L2) is recirculated to the second distillation apparatus of the thermal dissociation unit.

23. The process according to claim 15, wherein the first bottoms mixture (S1) obtained in step (c) or optionally the lower phase (L2) is recirculated to the third phase separation apparatus of the thermal dissociation unit.

24. The process according to claim 15, wherein the bottoms mixture (S1) obtained in step (c) is worked up further according to step (d) and the upper phase (U2) is recirculated to the extraction unit in step (b2) and the lower phase (L2) is fed to the thermal dissociation unit in step (e).

25. The process according to claim 15, wherein a tertiary amine of the general formula (A1) in which the radicals R¹, R², R³ are selected independently from the group consisting of C₅-C₆-alkyl, C₅-C₈-cycloalkyl, benzyl and phenyl is used as tertiary amine.

26. The process according to claim 25, wherein tri-n-hexylamine is used as tertiary amine (A1).

27. The process according to claim 15, wherein water, methanol or a mixture of water and methanol is used as polar solvent.

28. The process according to claim 15, wherein the upper phase (U3) is thermally treated at from 100 to 200° C. before being recirculated to the hydrogenation reactor in order to reactivate the inhibited complex catalyst.
